SEQUENCE LISTING (1 sequence)

SEQ ID NO: 1 - DNA, 357 bp, Unknown
  Arabidopsis sp. and lambda phage
agatatcgattcgtagtgtttaacatctgtgtaatttcttgcttgattgtgaaattaggattttcaaggacgatctattcaatttttgtgttttctttgttcgattctctctgttttaggtttcttatgtttagatccgtttctctttggagttgttttgatttctcttacggcttttgatttggtatatgttcgctgattggtttctacttgttctattgttttatttcaggttgaagcctgcttttttatactaacttgagcgaatccggattaggatccgtcgacactagtgaaaggagataggatccaagcttggcgtaatcatggtcatagctgtttcctgtgtgaaattgt

(19) United States
(12) Patent Application Publication
(10) Pub. No.: US 2010/0184128 A1
(43) Pub. Date: Jul. 22, 2010

(54) PRODUCTION OF PRODUCTS OF PHARMACEUTICAL INTEREST IN PLANT CELL CULTURES

(75) Inventors: William M. Ainley, Carmel, IN (US); Steven R. Webb, Westfield, IN (US); Michael Lindenbaum, Beaconsfield (CA); Steven Fabijanski, Orleans (CA); Ping Fu, Saskatoon (CA); Elizabeth-France Marillia, Asquith (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: DOW AGROSCIENCES LLC, Indianapolis, IN (US)

(21) Appl. No.: 12/571,086

(22) Filed: Sep. 30, 2009

Related U.S. Application Data
(60) Provisional application No. 61/102,528, filed on Oct. 3, 2008.

(51) Int. Cl. C12P 1/00; C12N 15/82; C12N 5/10

(52) U.S. Cl. 435/41; 435/468; 435/412; 435/414; 435/415; 435/416; 435/419

(57) ABSTRACT

The present invention relates to transgenic plant cell cultures comprising transgenic plant cells comprising a plurality of nucleic acids heterologous to said plant, each of said nucleic acids comprising a coding sequence encoding a pharmaceutical product of interest operably linked to one or more regulatory elements for directing expression of said coding sequence in said plant cell, said nucleic acids being stably integrated at or adjacent to native rDNA of said plant cell; methods of producing the transgenic plant cell culture; and methods of producing a pharmaceutical product of interest using the transgenic plant cell culture.

### CROSS-REFERENCE TO RELATED APPLICATIONS

This application claims benefit of and priority from U.S. Provisional Application No. 61/102,528 filed on Oct. 3, 2008, which is incorporated herein by reference.

### FIELD OF THE INVENTION

The present invention relates generally to the fields of plant cell culture and protein production in plant cell cultures. In particular, the invention relates to the production of products of pharmaceutical interest in plant cell cultures.

### BACKGROUND OF THE INVENTION

Technology for the fermentation of plant cell cultures has been in place for decades for production of plant secondary metabolites such as shikonin, ginsenoside, rosmarinic acid (reviewed in Mulabagal and Tsay. International Journal of Applied Science and Engineering, 2: 29-48, 2004) and the widely known drug paclitaxel (Tabata. Curr Drug Targets, 7: 453-461, 2006). Further, plant cell cultures have been used to express exogenous proteins including IL-2 and IL-4 (Magnuson et al. Protein Exp Pur, 13: 45-52, 1998) GM-CSF (James et al. Protein Exp Pur, 19: 131-134, 2000) and antibodies (Tsoi and Doran. Biotechnol Appl Biochem, 35: 171-180, 2002) among others.

The widespread commercial utility of plant cell culture-based production technology requires demonstration of a number of benchmarks, foremost of which is stable gene expression, accumulation of biologically active, appropriately processed protein and maintenance of productivity over time and at increasing scale (Tsoi and Doran. Biotechnol Appl Biochem, 35: 171-180, 2002; Sharp and Doran. Biotechnol Prog, 17: 979-992, 2001; James and Lee. Plant Cell Reports, 25: 723-727, 2006).

Stram et al (U.S. Pat. No. 6,528,063) and Wu et al (Avian Dis, 48: 663-668, 2004) describe the expression of IBDV particles in plant cells by Agrobacterium mediated gene transfer, where the particles are for use as vaccines in poultry. The above reports relied on plant transformation using Agrobacterium, with its inherent limitations, such as low copy number random integration of the transgene into the plant cell genome.

### SUMMARY OF THE INVENTION

In one aspect, the invention provides a method for producing a transgenic plant cell culture, comprising:

(a) co-transforming plant cells with:

i. a first nucleic acid, said first nucleic acid comprising a nucleotide sequence of at least contiguous 100 nucleotides, said nucleotide sequence possessing at least 50% sequence identity over its entire length to a native ribosomal DNA (rDNA) sequence of said plant cells; and

ii. a second nucleic acid, said second nucleic acid comprising a coding sequence operably linked to one or more regulatory elements for directing expression of said coding sequence in said plant cells, said coding sequence encoding a pharmaceutical product of interest;

thereby obtaining transgenic plant cells;
(b) culturing a plurality of said transgenic plant cells;
(c) selecting and isolating from said plurality of transgenic plant cells transgenic plant cells wherein said second nucleic acid is stably integrated into or adjacent to native rDNA of said transgenic plant cells and wherein said second nucleic acid is amplified, resulting in said transgenic plant cell culture.

In another aspect, the invention provides a transgenic plant cell culture produced by the method described above.

In another aspect, the invention provides a transgenic plant cell culture comprising transgenic plant cells comprising a plurality of nucleic acids heterologous to said plant, each of said nucleic acids comprising a coding sequence encoding a pharmaceutical product of interest operably linked to one or more regulatory elements for directing expression of said coding sequence in said plant cell, said nucleic acids being stably integrated at or adjacent to native rDNA of said plant cell.

In another aspect, the invention provides a plant cell obtained from a plant cell culture as described above.

In another aspect, the invention provides a method for producing a pharmaceutical product of interest, said method comprising:

(a) culturing a transgenic plant cell culture as described above under conditions sufficient for expression of said pharmaceutical product of interest from said coding sequence; and,
(b) recovering said pharmaceutical product of interest.

Other aspects and features of the present invention will become apparent to those of ordinary skill in the art upon review of the following description of specific embodiments of the invention in conjunction with the accompanying figures.

### BRIEF DESCRIPTION OF DRAWINGS

FIG. 1: A. Cloning strategy for the core pV2 vector. B. Nucleic acid constructs used for plant cell transformation. C. Steps in the transformation of plant cells.

FIG. 2: A. Analysis of VP2 in DB 14 and DB 16 series plant cell transformation events by mini-culture assay and ELISA. The cells were transformed with rDNA/core pV2 except event 1060-199, which was transformed by Agrobacterium mediated gene transfer.

FIG. 3: Southern analysis of pV2 in transformed cells. A. (left panel) DNA was digested with XbaI (X in panel c) and the blots probed with a fragment corresponding to the IBDV E91 VP2 gene (black bar above VP2 in panel c). Right panel, copy number reconstructions were done using Xba digested pV2 and diluting the DNA to equal the indicated copy number if present in genomic DNA. B. Same analysis as described in panel a, but the DNA was digested with PacI. C. Structure of pV2 insert. Arrows indicate the coding sequences for IBDV E91 VP2 (grey) and the PAT (black) genes. X and P represent restriction sites for XbaI and PacI, respectively. The two headed arrows indicate the expected size of the fragments containing VP2 when pV2 is digested with XbaI or PacI.

FIG. 4: FISH analysis of VP2 transgenic plant cells before and after extended culture. Representative metaphase spreads from rDNA/core pV2 transformation events 14-46 (panels A and B), 16-37 (panels C and D) and 16-40 (panels E and F) were hybridized with an rDNA gene probe (rhodamine label) and a VP2 probe (FITC label) either before (panels A, B and C) or after (panels D, E and F) 11 culture passages. VP2 that has integrated into chromosomes are indicated by arrows. Higher magnification of rDNA and VP2 double-labeled or DAPI labeled chromosomes are shown in the insets. FIG. 4, panel G shows wild-type BY2 cells stained with rDNA and VP2 probes.

FIG. 5: A. Stability of VP2 expression in cell lines over 7 culture passages. VP2 from transformation events harvested at Day 14 post-subculturing, over the interval of passage 4 through 10, were assessed by ELISA. The average and percent coefficient of variance are indicated. B. Western blot analysis to compare VP2 expressed from rDNA/core pV2 transformed cells (lanes 5-7) and cells transformed by Agrobacterium (lanes 2-4). Lane 1 contains inactivated IBDV. All lanes contain the same amount of total soluble protein. C. Evaluation of insert stability between passage 4 (p4) and passage 11 (p11) in independent lineages (indicated as a, b or c) of transformation events 14-46, 16-40, and 16-37. D. Serum neutralization inhibition assay results for passage 4 samples of cells transformed with rDNA/core pV2, Agrobacterium mediated VP2 transfer, or controls.

### DETAILED DESCRIPTION OF INVENTION

The production of proteins using plant cell cultures offers potential as a safe source of protein products as it is unlikely to be contaminated with mammalian pathogens and adventitious agents, as in animal cell cultures.

The present invention is based on targeted integration of a product gene into or adjacent an rDNA array of a plant cell chromosome. As a result, concomitant amplification of both the inserted genes and the rDNA arrays occurs, leading to stable heterochromatic DNA

The protein production system of the present invention comprises the integration of the inserted gene into genomic DNA regions capable of supporting high level gene expression. The site of gene insertion may be considered an “Engineered Trait Loci” or “ETL”.

In the context of the present invention, there is provided a first nucleic acid comprising a sequence that is homologous to native rDNA. Alternatively, the first nucleic acid consists or consists essentially of a sequence that is homologous to native rDNA. rDNA may be organized as arrays and are regions of high transcription and high stability that are amenable to the integration of one or more nucleic acids encoding a pharmaceutical product of interest into the plant genome. Integration may occur at one or more sites. The present invention comprises the selection of transformed cells in which one or a plurality of the second nucleic acids has integrated at or adjacent one or more rDNA arrays. Further, the produced product of interest may be highly expressed and biologically active.

The genes encoding cytosolic ribosomal RNA such as 18S, 5.8S, 26S and 5S subunits are generally organized in arrays in higher eukaryotes, the repeated unit of which contains the transcription unit and a spacer sequence. The genes encoding 18S, 5.8S and 26S ribosomal subunits are transcribed as a single unit of 45S. The 5S rDNA gene is also arranged in clusters of tandemly repeated units. In higher eukaryotes, 5S and 18S-5.8S-26S rRNA genes are organized in separate clusters. rDNA arrays may be localized on either a single or several chromosomes and may be pericentric or non-pericentric.

rDNA arrays are highly variable in size and location in plant genomes (Raina and Mukai. Genome, 42: 52-59, 1999). For example, soybean had only one 5S and one 45S rDNA locus whereas common bean had more than two 5S rDNA loci and two 45S rDNA loci (Shi et al. Theoretical and Applied Genetics, 93: 136-141, 1996). rDNA arrays are highly transcribed regions of the genome (Tsang et al. EMBO J, 26: 448-458, 2007).

In the context of the present invention, the first nucleic acid may be homologous to native rDNA. As used herein, “homologous” refers to two sequences that show at least 50%, at least 55%, at least 60%, at least 65%, at least 70%, at least 75%, at least 80%, at least 83%, at least 85%, at least 87%, at least 89%, at least 91%, at least 93%, at least 95%, at least 97%, or at least 99% sequence identity over its entire length to a native ribosomal DNA (rDNA) of the plant cell to be transformed. Typically, homologous sequences have at least 50% sequence identity.

The first and second nucleic acids are typically introduced into cells at a ratio of first to second nucleic acid of 300:1, 200:1, 100:1, 50:1, 30:1, 25:1, 20:1, 15:1, 10:1, 9:1, 8:1, 7:1, 6:1, 5:1, 4:1, 3:1, 2:1, or 1:1, and preferably at a ratio of 10:1.

As used herein, a “construct” refers to any recombinant polynucleotide molecule such as a plasmid, cosmid, virus, vector, autonomously replicating polynucleotide molecule, phage, or linear or circular single-stranded or double-stranded DNA or RNA polynucleotide molecule, derived from any source.

The first nucleic acid comprises, consists of, or consists essentially of a nucleotide sequence that has at least 50%, at least 55%, at least 60% at least 65%, at least 70%, at least 75%, at least 80%, at least 83%, at least 85%, at least 87%, at least 89%, at least 91%, at least 93%, at least 95%, at least 97%, at least 99% or has 100% sequence identity over its entire length to a native ribosomal DNA (rDNA) of the plant cell to be introduced. Typically, the first nucleic acid comprises, consists of or consists essentially of a nucleotide sequence possessing at least 50% sequence identity over its entire length to a native rDNA sequence. As used herein, “consists essentially of” or “consisting essentially of” means that the nucleic acid sequence includes one or more nucleotide bases, included within the sequence or at one or both ends of the sequence, but that the additional nucleotide bases do not materially affect the function of the nucleic acid sequence.

The first nucleic acid comprises, consists of, or consists essentially of a nucleotide sequence that is 5S, 5.8S, 18S or 26S rDNA.

The first nucleic acid typically comprises a nucleotide sequence that is at least 100, at least 125, at least 150, at least 250, at least 500, at least 750, at least 1000, at least 1250, at least 1500, at least 1750, at least 2000, at least 2500, at least 3000, at least 5000, or at least 10000 nucleotides or base pairs in length; and preferably a nucleotide sequence that is 1.7-2.8 kb in length.

The second nucleic acid comprises a coding sequence encoding a pharmaceutical product of interest.

The first and/or second nucleic acids typically integrate into or adjacent to rDNA. The nucleic acid may integrate into or adjacent to pericentric and/or non-pericentric rDNA. In one embodiment, one or more copies of the second nucleic acid may integrate into one or more regions of pericentric and/or non-pericentric rDNA. As used herein, the term “pericentric” means immediately adjacent to or close to the centromere of a chromosome. The term “telocentric” means immediately adjacent to or close to the telomere of a chromosome. The nucleic acid may integrate into rDNA at a position that is closer to the telomere than to the centromere, or that is closer to the centromere than to the telomere, or both.

In one embodiment, the first and second nucleic acids are on the same construct. In another embodiment, the first and second nucleic acids are on separate constructs.

One or more copies of the first and/or second nucleic acid may integrate into or adjacent to native rDNA. First and/or second nucleic acids may be amplified at the site of the insertion to produce multiple copies in low or relatively low copy number (e.g. 2 to 100 copies). The inserted and/or amplified first and/or second nucleic acids may be in sufficiently close proximity that they segregate as a single genetic locus.

The integration of the heterologous DNA into or adjacent the rDNA array may result in a continuum of event structures including, but not limited to, a single insert without any duplication, an insert that is duplicated within a very localized duplication region, and an insert that undergoes large scale amplification that provides gross chromosomal changes such as “sausage chromosomes” with many millions of amplified and duplicated sequences. In one embodiment, the integrated heterologous DNA is an insert that is duplicated or at low copy number, and without gross cytomorphological chromosomal events.

The present invention relates to methods of producing a transgenic plant cell culture comprising a first and a second nucleic acid.

In the context of the present invention, “plant cell culture” refers to a collection of cells of plant origin, whether unicellular or in aggregate form, which are capable of being maintained, expanded, propagated and otherwise grown and cultured in suitable growth medium. The terms “plant cell line”, “plant tissue culture”, and “callus” are use synonymously in this specification. Plant cell cultures are typically grown as cell suspension cultures in liquid medium or as callus cultures on solid medium.

As used herein, “transgenic plant cell culture” refers to a plant cell culture or progeny thereof wherein foreign genetic material has been introduced into the genome of the plant cell within the plant cell culture. The terms “transgenic plant cell culture” and “transformed plant cell culture” are used synonymously to refer to a plant cell culture whose genome contains exogenous genetic material.

As used herein, “nucleotide sequence” or “nucleic acid” refers to a polymer of DNA or RNA which can be single or double stranded and optionally containing synthetic, non-natural or altered nucleotide bases capable of incorporation into DNA or RNA polymers. “Nucleic acids” or “Nucleic acid sequences” may encompass genes, cDNA, DNA and RNA encoded by a gene. Nucleic acids or nucleic acid sequences may comprise at least 3, at least 10, at least 100, at least 1000, at least 5000, or at least 10000 nucleotides or base pairs.

Nucleic acids may be modified by any chemical and/or biological means known in the art including, but not limited to, reaction with any known chemicals such as alkylating agents, browning sugars, etc; conjugation to a linking group (e.g. PEG); methylation; oxidation; ionizing radiation; or the action of chemical carcinogens. Such nucleic acid modifications may occur during synthesis or processing or following treatment with chemical reagents known in the art.

As used herein, “% sequence identity” is determined by comparing two optimally aligned sequences over a comparison window, where the fragment of the polynucleotide sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid residue occurs in both sequences to yield the number of matched positions, dividing the number of matched positions by the total number of positions in the comparison window and multiplying the result by 100 to provide the percentage of sequence identity. Algorithms to align sequences are known in the art. Exemplary algorithms include, but are not limited to, the local homology algorithm of Smith and Waterman (Add APL Math, 2: 482, 1981); the homology alignment algorithm of Needleman and Wunsch (J Mol Biol, 48: 443, 1970); the search for similarity method of Pearson and Lipman (Proc Natl Acad Sci USA, 85: 2444, 1988); and computerized implementations of these algorithms (GAP, BESTFIT, BLAST, PASTA, and TFASTA in the Wisconsin Genetics Software Package, Genetics Computer Group (GCG), 575 Science Dr., Madison, Wis.). In one aspect, two sequences may be aligned using the “Blast 2 Sequences” tool at the NCBI website at default settings (Tatusova and Madden. FEMS Microbiol Lett, 174: 247-250, 1999). Alternatively, nucleic acids sequences may be aligned by human inspection.

As used herein, “native ribosomal DNA” refers to the ribosomal DNA that naturally occurs in the cell that is to be transformed.

As used herein, “rDNA” means ribosomal DNA and refers to genes encoding ribosomal RNA including, but not limited to, genes encoding the 5S, 5.8S, 18S and 25/26S ribosomal RNA.

As used herein, “coding sequence” refers to a DNA or RNA sequence that codes for a specific amino acid sequence and may exclude the non-coding sequences such as introns and untranslated regions of a gene. The coding sequence may be any length. A coding sequence may comprise at least 6, at least 10, at least 100, at least 1000, at least 5000, or at least 10000 nucleotides or base pairs.

As used herein, “operably-linked” refers to two nucleic acid sequences that are related physically or functionally. For example, a regulatory element is said to be “operably linked to” to a coding sequence if the two sequences are situated such that the regulatory DNA sequence affects expression of the coding DNA sequence. Coding sequences may be operably-linked to regulatory sequences in sense or antisense orientation.

As used herein, “regulatory element” refers nucleic acid sequences that affect the expression of a coding sequence. Regulatory elements are known in the art and include, but are not limited to, promoters, enhancers, transcription terminators, polyadenylation sites, matrix attachment regions and/or other elements that regulate expression of a coding sequence.

As used herein, a “promoter” refers to a nucleotide sequence that directs the initiation and rate of transcription of a coding sequence (reviewed in Roeder, Trends Biochem Sci, 16: 402, 1991). The promoter contains the site at which RNA polymerase binds and also contains sites for the binding of other regulatory elements (such as transcription factors). Promoters may be naturally occurring or synthetic (see Datla et al. Biotech Ann. Rev 3:269, 1997 for review of plant promoters). Further, promoters may be species specific (for example, active only in B. napus); tissue specific (for example, the napin, phaseolin, zein, globulin, dlec2, γ-kafirin seed specific promoters); developmentally specific (for example, active only during embryogenesis); constitutive (for example maize ubiquitin, rice ubiquitin, rice actin, Arabidopsis actin, sugarcane bacilliform virus, CsVMV and CaMV 35S, Arabidopsis polyubiquitin, Agrobacterium tumefaciens-derived nopaline synthase, octopine synthase, and mannopine synthase gene promoters); or inducible (for example the stilbene synthase promoter and promoters induced by light, heat, cold, drought, wounding, hormones, stress and chemicals). A promoter includes a minimal promoter that is a short DNA sequence comprised of a TATA box or an lnr element, and other sequences that serve to specify the site of transcription initiation, to which regulatory elements are added for control of expression. A promoter may also refer to a nucleotide sequence that includes a minimal promoter plus DNA elements that regulates the expression of a coding sequence, such as enhancers and silencers.

As used herein, “expression” or “expressing” refers to production of any detectable level of a product encoded by the coding sequence

Enhancers and silencers are DNA elements that affect transcription of a linked promoter positively or negatively, respectively (reviewed in Blackwood and Kadonaga, Science, 281: 61, 1998).

Polyadenylation site refers to a DNA sequence that signals the RNA transcription machinery to add a series of the nucleotide A at about 30 bp downstream from the polyadenylation site.

Transcription terminators are DNA sequences that signal the termination of transcription. Transcription terminators are known in the art. The transcription terminator may be derived from Agrobacterium tumefaciens, such as those isolated from the nopaline synthase, mannopine synthase, octopine synthase genes and other open reading frame from Ti plasmids. Other terminators may include, without limitation, those isolated from CaMV and other DNA viruses, dlec2, zein, phaseolin, lipase, osmotin, peroxidase, and PinII genes.

In the context of the present invention, the coding sequence encodes a pharmaceutical product of interest.

As used herein, a “pharmaceutical product of interest” includes, but is not limited to, enzymes, toxins, cell receptors, ligands, viral or bacterial proteins or antigens, signal transducing agents, cytokines, antibodies and growth factors. The pharmaceutical product of interest may include proteins, peptides, or fragments thereof. Modifications of the product of interest may include in vivo and in vitro chemical derivatization of polypeptides, e.g., acetylation, carboxylation, phosphorylation, or glycosylation; such modifications may occur during polypeptide synthesis or processing or following treatment with isolated modifying enzymes.

As used herein, the terms “peptide”, “oligopeptide”, “polypeptide” and “protein” may be used interchangeably. Peptides may contain non-natural amino acids and may be joined to linker elements known to the skilled person. Peptides may also be monomeric or multimeric. Peptide fragments comprise a contiguous span of at least 5, at least 10, at least 25, at least 50, at least 100, at least 250, at least 500, at least 1000, at least 1500, or at least 2500 consecutive amino acids and may retain the desired activity of the full length peptide.

The pharmaceutical product of interest may be any useful protein-based therapeutic or prophylactic agent. A preferred group of protein therapeutic or prophylactic agents may include, but are not limited to, vaccine antigens that are useful for disease prevention. As used herein, the term “antigen” refers to any proteinaceous substance that elicits an immune response, either antibody or cellular, in animals. Vaccine antigens are well know in the art and are consistent with the instant invention. Exemplary antigens include, but are not limited to, the HA (hemagglutinin) protein of AIV (Avian Influenza Virus); the HN (hemagglutinin/neuraminidase) protein of avian Newcastle Disease Virus; VP2, of infectious bursal disease virus (IBDV); an enzyme ADP ribosyl transferase (LT-A subunit of heat labile toxin of E. coli); a bacterial toxin LT of E. coli; and proteins derived from human viruses including, but not limited to, poliovirus, human rhinovirus, hepatitis A virus, human immunodeficiency virus, human influenza, human papillomavirus, herpes simplex virus, picornaviruses such as foot-and-mouth disease virus, Dengue and West Nile viruses and respiratory syncytial virus. In one aspect of the invention, the pharmaceutical product of interest is VP2 or fragments thereof.

In another aspect, the pharmaceutical product of interest is a cytokine. Cytokines are proteins made by cells that affect the behaviour of other cells. Cytokines are known in the art and include, but are not limited to, interleukins, interferons, hematopoetins, chemokines, and TNF family proteins.

In a further aspect, the pharmaceutical product of interest is an antibody. Antibodies are plasma proteins that bind specifically to particular molecules known as antigens and are produced in response to immunization with an antigen. Each antibody molecule has a unique structure that allows it to bind to its specific antigen, but all antibodies have the same basic structure and are collectively called immunoglobulins.

As used herein, “antibody” includes monoclonal antibodies (including full length monoclonal antibodies), polyclonal antibodies, multispecific antibodies (e.g., bispecific antibodies), single domain antibodies and antibody fragments. “Antibody fragments” comprise a portion of a full length antibody, generally the antigen binding or variable region thereof. Examples of antibody fragments include Fab, Fab′, F(ab′)2, and Fv fragments; diabodies; linear antibodies; single-chain antibody molecules; and multi-specific antibodies formed from antibody fragments. The term “antibody” also includes chimeric or humanized antibodies.

The present invention is not limited to any particular method for transforming plant cells. Methods for introducing nucleic acids into cells (also referred to herein as “transformation”) are known in the art and include, but are not limited to: Viral methods (Clapp. Clin Perinatol, 20: 155-168, 1993; Lu et al. J Exp Med, 178: 2089-2096, 1993; Eglitis and Anderson. Biotechniques, 6: 608-614, 1988; Eglitis et al, Avd Exp Med Biol, 241: 19-27, 1988); physical methods such as microinjection (Capecchi. Cell, 22: 479-488, 1980), electroporation (Wong and Neumann. Biochim Biophys Res Commun, 107: 584-587, 1982; Fromm et al, Proc Natl Acad Sci USA, 82: 5824-5828, 1985; U.S. Pat. No. 5,384,253) and the gene gun (Johnston and Tang. Methods Cell Biol, 43: 353-365, 1994; Fynan et al. Proc Natl Acad Sci USA, 90: 11478-11482, 1993); chemical methods (Graham and van der Eb. Virology, 54: 536-539, 1973; Zatloukal et al. Ann NY Acad Sci, 660: 136-153, 1992); and receptor mediated methods (Curie) et al. Proc Natl Acad Sci USA, 88: 8850-8854, 1991; Curiel et al. Hum Gen Ther, 3: 147-154, 1992; Wagner et al. Proc Natl Acad Sci USA, 89: 6099-6103, 1992).

The introduction of DNA into plant cells by Agrobacterium mediated transfer is well known to those skilled in the art. Virulent strains of Agrobacterium contain a large plasmid DNA known as a Ti-plasmid that contains genes required for DNA transfer (vir genes), replication and a T-DNA region that is transferred to plant cells. The T-DNA region is bordered by T-DNA border sequences that are essential to the DNA transfer process. These T-DNA border sequences are recognized by the vir genes. The two primary types of Agrobacterium-based plant transformation systems include binary [see for example U.S. Pat. No. 4,940,838] and co-integrate [see for example Fraley et al. Biotechnology, 3: 629-635, 1985] methods. In both systems, the T-DNA border repeats are maintained and the natural DNA transfer process is used to transfer the DNA fragment located between the T-DNA borders into the plant cell genome.

Another method for introducing DNA into plant cells is by biolistics. This method involves the bombardment of plant cells with microscopic particles (such as gold or tungsten particles) coated with DNA. The particles are rapidly accelerated, typically by gas or electrical discharge, through the cell wall and membranes, whereby the DNA is released into the cell and incorporated into the genome of the cell. This method is used for transformation of many crops, including corn, wheat, barley, rice, woody tree species and others. Biolistic bombardment has been proven effective in transfecting a wide variety of animal tissues as well as in both eukaryotic and prokaryotic microbes, mitochondria, and microbial and plant chloroplasts (Johnston. Nature, 346: 776-777, 1990; Klein et al. Bio/Technol, 10: 286-291, 1992; Pecorino and Lo. Curr Biol, 2: 30-32, 1992; Jiao et al, Bio/Technol, 11: 497-502, 1993).

Another method for introducing DNA into plant cells is by electroporation. This method involves a pulse of high voltage applied to protoplasts/cells/tissues resulting in transient pores in the plasma membrane which facilitates the uptake of foreign DNA. The foreign DNA enter through the holes into the cytoplasm and then to the nucleus.

Plant cells may be transformed by liposome mediated gene transfer. This method refers to the use of liposomes, circular lipid molecules with an aqueous interior, to deliver nucleic acids into cells. Liposomes encapsulate DNA fragments and then adhere to the cell membranes and fuse with them to transfer DNA fragments. Thus, the DNA enters the cell and then to the nucleus.

Other well-know methods for transforming plant cells which are consistent with the present invention include, but are not limited to, pollen transformation (See University of Toledo 1993 U.S. Pat. No. 5,177,010); Whiskers technology (See U.S. Pat. Nos. 5,464,765 and 5,302,523).

The nucleic acid constructs of the present invention may be introduced into plant protoplasts. Plant protoplasts are cells in which its cell wall is completely or partially removed using either mechanical or enzymatic means, and may be transformed with known methods including, calcium phosphate based precipitation, polyethylene glycol treatment and electroporation (see for example Potrykus et al., Mol. Gen. Genet., 199: 183, 1985; Marcotte et al., Nature, 335: 454, 1988). Polyethylene glycol (PEG) is a polymer of ethylene oxide. It is widely used as a polymeric gene carrier to induce DNA uptake into plant protoplasts. PEG may be used in combination with divalent cations to precipitate DNA and effect cellular uptake. Alternatively, PEG may be complexed with other polymers, such as poly(ethylene imine) and poly L lysine.

The introduction of nucleic acids into a sample of plant cells results in a transformation event. As used herein, “transformation event” or “event” refers to one instance of plant cell transformation. These terms may also refer to the outcome of one sample of plant cells transformed with one of the transformation methods described herein.

In the context of the present invention, the nucleic acids are heterologous DNA, that is, introduced into the cell. As used herein, “heterologous”, “foreign” and “exogenous” DNA and RNA are used interchangeably and refer to DNA or RNA that does not occur naturally as part of the plant genome in which it is present or which is found in a location or locations in the genome that differ from that in which it occurs in nature. Thus, heterologous or foreign DNA or RNA is nucleic acid that is not normally found in the host genome in an identical context. It is DNA or RNA that is not endogenous to the cell and has been exogenously introduced into the cell. In one aspect, heterologous DNA may be the same as the host DNA but modified by methods known in the art, where the modifications include, but are not limited to, insertion in a vector, linked to a foreign promoter and/or other regulatory elements, or repeated at multiple copies. In another aspect, heterologous DNA may be from a different organism, a different species, a different genus or a different kingdom, as the host DNA. Further, the heterologous DNA may be a transgene. As used herein, “transgene” refers to a segment of DNA containing a gene sequence that has been isolated from one organism and introduced into a different organism.

The use of plant cells for producing transgenic plants is known in the art. Suitable plant cells may be obtained from a number of plants that include, but are not limited to, borage, canola, castor, corn, cotton, Crambe spp., flax, nasturtium, olive, palm, peanut, rapeseed, rice, soybean, and sunflower. Preferably the plant cell cultures useful in the present invention are derived from corn, rice or tobacco plants. Tobacco suspension cell cultures such NT-I and BY-2 (An. Plant Physiol, 79: 568-570, 1985; Nagata et al, Int Rev Cytol, 132: 1-30, 1992) are particularly susceptible to handling in culture, readily transformed, produce stably integrated events and amenable to cryopreservation.

Plant cell culture techniques are known in the art (see for example Fischer et al. Biotechnol Appl Biochem, 30: 109-112, 1999; Doran. Current Opinions in Biotechnology, 11: 199-204, 2000). The skilled person would appreciate that the composition of the culture media, its pH and the incubating conditions, such as temperatures, aeration, CO₂ levels, and light cycles, may vary depending on the type of cells.

After transformation, plant cells may be sub-cloned to obtain clonal populations of cells. Methods of sub-cloning cells are known in the art and include, but are not limited to, limiting dilution of the pool of transformed cells. The transformed cells may also be grown under selective pressure to identify those that contain and/or express the pharmaceutical product of interest. In this regard, the nucleic acids encodes a selectable marker. Selectable markers may be used to select for plants or plant cells that contain the exogenous genetic material. The exogenous genetic material may include, but is not limited to, an enzyme that confers resistance to an agent such as a herbicide or an antibiotic, or a protein that reports the presence of the construct.

Numerous plant selectable marker systems are known in the art and are consistent with this invention. The following review article illustrates these well known systems: Miki and McHugh; Journal of Biotechnology 107: 193-232; Selectable marker genes in transgenic plants: applications, alternatives and biosafety (2004).

Examples of a selectable marker include, but are not limited to, a neo gene, which codes for kanamycin resistance and can be selected for using kanamycin, NptII, G418, hpt etc.; an amp resistance gene for selection with the antibiotic ampicillin; an hygromycinR gene for hygromycin resistance; a BAR gene (encoding phosphinothricin acetyl transferase) which codes for bialaphos resistance including those described in WO/2008/070845; a mutant EPSP synthase gene, aadA, which encodes glyphosate resistance; a nitrilase gene, which confers resistance to bromoxynil; a mutant acetolactate synthase gene (ALS), which confers imidazolinone or sulphonylurea resistance, ALS, and a methotrexate resistant DHFR gene.

Further, screenable markers that may be used in the context of the invention include, but are not limited to, a β-glucuronidase or uidA gene (GUS), which encodes an enzyme for which various chromogenic substrates are known, green fluorescent protein (GFP), and luciferase (LUX).

A nucleic acid of the present invention may encode a gene conferring resistance to bialaphos (also known as bilanafos or PPT; commercialized under the trade-marks Basta®, Buster® and Liberty®) which is converted to the phytotoxic agent phosphinothricin in plant cells. In one aspect, the bialaphos resistance gene is a BAR gene. In another aspect, multiple copies of the glutamine synthetase gene confer resistance to bialaphos.

After preparing clonal populations of transgenic plant cells, the cells may be characterized and selected based on analysis at the level of DNA, RNA and protein. Preferably, transgenic plant cells in which the nucleic acid construct is stably integrated into or adjacent rDNA are selected. As used herein, “stably integrated” refers to the integration of genetic material into the genome of the transgenic plant cell and remains part of the plant cell genome over time. Thus a cell comprising a stably integrated nucleic acid construct of the present invention would continue to produce the pharmaceutical product of interest.

Stable integration of nucleic acid constructs may be influenced by a number of factors including, but not limited to, the transformation method used and the vector containing the gene of interest. The transformation method determines which cell type can be targeted for stable integration. The type of vector used for stable integration defines the integration mechanism, the regulation of transgene expression and the selection conditions for stably expressing cells. After integration, the level and time of expression of the gene of interest may depend on the linked promoter and on the particular integration site.

The site of integration may affect the transcription rate of the gene of interest. Usually an expression plasmid is integrated into the genome of the target cell randomly. Integration into inactive heterochromatin results in little or no transgene expression, whereas integration into active euchromatin often allows transgene expression.

In the context of the present invention, the first and/or second nucleic acids target the native rDNA arrays of the plant cell to be transformed. Thus the first nucleic acid comprising rDNA sequences homologous to native rDNA may be integrated at or adjacent to the native rDNA array. Further, the second nucleic acid comprising a coding sequence operably linked to one or more regulatory sequences may be integrated at or adjacent to the native rDNA array, wherein the coding sequences encodes one or more pharmaceutical products of interest.

Following transformation, cells in which the nucleic acid construct is integrated into rDNA are selected. As noted herein, rDNA arrays are regions of active transcription; thus, the gene encoding the pharmaceutical product of interest is expressed.

The integrated first and/or second nucleic acids may be present in the transgenic plant cell in 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies, 40 copies, 41 copies, 42 copies, 43 copies, 44 copies, 45 copies, 46 copies, 47 copies, 48 copies, 49 copies, 50 copies, 51 copies, 52 copies, 53 copies, 54 copies, 55 copies, 56 copies, 57 copies, 58 copies, 59 copies, 60 copies, or more.

Targeted introduction of DNA into the genome may be accomplished by a number of methods including, but not limited to, targeting recombination, homologous recombination and site-specific recombination (see review Baszcynski et al. Transgenic Plants, 157: 157-178, 2003 for review of site-specific recombination systems in plants). Homologous recombination and gene targeting in plants (reviewed in Reiss. International Review of Cytology, 228: 85-139, 2003) and mammalian cells (reviewed in Sorrell and Kolb. Biotechnology Advances, 23: 431-469, 2005) are known in the art.

As used herein, “targeted recombination” refers to integration of a heterologous nucleic acid construct into a rDNA array, where the integration is facilitated by heterologous rDNA that is homologous to the native rDNA of the cell to be transformed.

Homologous recombination relies on sequence identity between a piece of DNA that is introduced into a cell and the cell's genome. Homologous recombination is an extremely rare event in higher eukaryotes. However, the frequency of homologous recombination may be increased with strategies involving the introduction of DNA double-strand breaks, triplex forming oligonucleotides or adeno-associated virus.

As used herein, “site-specific recombination” refers to the enzymatic recombination that occurs when at least two discrete DNA sequences interact to combine into a single nucleic acid sequence in the presence of the enzyme. Site-specific recombination relies on enzymes such as recombinases, transposases and integrases, which catalyse DNA strand exchange between DNA molecules that have only limited sequence homology. Mechanisms of site specific recombination are known in the art (reviewed in Grindley et al. Annu Rev Biochem, 75: 567-605, 2006). The recognition sites of site-specific recombinases (for example Cre and att sites) are usually 30-50 bp. The pairs of sites between which the recombination occurs are usually identical, but there are exceptions e.g. attP and attB of λ integrase (Landy. Ann Rev Biochem, 58: 913-949, 1989).

The nucleic acid construct of the present invention may comprise a site-specific recombination sequence. Site-specific recombination sequences may be useful for the directed integration of subsequently introduced genetic material into the transformed cell.

Preferably, the site-specific recombination sequence is an att sequence, for example, an att from λ phage. λ phage is a virus that infects bacteria. The integration of λ phage takes place at an attachment site in the bacterial genome, called att^{λ}. The sequence of the att site in the bacterial genome is called attB and consists of the parts B-O-B′, whereas the complementary sequence in the circular phage genome is called attP and consists of the parts P-O-P′. Integration proceeds via a Holliday structure and requires both the phage protein int and the bacterial protein IHF (integration host factor). Both int and IHF bind to attP and form an intrasome (a DNA-protein-complex) for site-specific recombination of the phage and host DNA. The integrated host and phage sequences become B-O-P′ - - - phage DNA - - - P-O-B′. Accordingly, att sites may be used for targeted integration of heterologous DNA.

Other site-specific recombination systems include, but are not limited to, Cre/Lox, FLP/FRT (For example, see Lyznik, et al., Site-Specific Recombination for Genetic Engineering in Plants, Plant Cell Rep, 21:925-932, 2003 and WO 99/25821.), PhiC31 att sites, Zinc Finger based systems, see inter alia U.S. Pat. No. 6,785,613.

Methods for identifying the presence of, or localizing, the nucleic acid construct within the transformed plant cell genome are known in the art and include, but are not limited to, fluorescence in situ hybridization (FISH) and PCR amplification followed by Southern blot analysis. In addition, the gene transcripts may be examined, for example, by Northern blot analysis or RT-PCR, while the pharmaceutical product of interest may be assessed, for example, by Western blot, Immuno histochemistry, enzyme assay, LC-MSMS, ELISA; and gas liquid chromatography. Further, the pharmaceutical product of interest may functionally assessed, for example, by serum neutralization inhibition assays; ligand binding assay.

Fluorescence in situ hybridization (FISH) is a molecular cytogenetic technique in which fluorescently labelled DNA probes are hybridized to metaphase spread or interphase nuclei. The sample DNA (metaphase chromosomes or interphase nuclei) is first denatured to separate the complementary strands within the DNA double helix structure. The fluorescently labelled probe of interest is then added to the denatured sample mixture and hybridized with the sample DNA at the target site as it re-anneals back into a double helix. The probe signal is assessed with a fluorescent microscope. A plurality of probes may be used to simultaneously co-localize distinct targets. In the context of the present invention, FISH may be used to co-localize the integrated nucleic acid constructs and the native rDNA array, thus identify transgenic plant cell lines that have a plurality of second nucleic acids integrated into rDNA.

Another method of identifying site of integration of the nucleic acid constructs of the present invention is by PCR followed by Southern blot analysis. The skilled person would appreciate that there are a number of PCR approaches to identifying the integrated nucleic acid construct. In one approach, one of the two primers corresponds to a sequence within the nucleic acid construct, and the other corresponds to a sequence adjacent to the nucleic acid. In another approach, one primer corresponds to a genomic DNA sequence that is upstream of a putative nucleic acid integration site and the other primer corresponds to a genomic DNA sequence that is downstream of the putative nucleic acid integration site. Subsequently, the PCR product is probed with a nucleic acid probe by Southern blot analysis. Polymerase chain reaction (PCR) (U.S. Pat. Nos. 4,683,195; 4,683,202; and 4,965,188) is used to increase the concentration of a target nucleic acid sequence in a sample without cloning, and requires the availability of target sequence information to design suitable forward and reverse oligonucleotide primers which are typically 10 to 30 base pairs in length. Southern blotting combines agarose gel electrophoresis for size separation of the amplified DNA with methods to transfer the size-separated DNA to a filter membrane for nucleic acid probe hybridization. The probe may be conjugated to a label, such as a radiolabel or a fluorescent label, so that the DNA/probe hybrid may be visualized, for example, on film or by a phosphoimager. Southern blots are a standard tool of molecular biologists (J. Sambrook et al. (1989) Molecular Cloning: A Laboratory Manual (Cold Spring Harbor Press, NY), pp 9.31-9.58). Southern blot analysis may also be used to determine the copy number of the nucleic acid construct that has integrated into the plant cell genome by comparing the quantity of the integrated nucleic acid construct with known quantities of DNA probed on the same blot. Methods of quantitating the detected DNA are known in the art, and include for example, densitometry.

As used herein, a “nucleic acid probe” is a DNA or RNA fragment that includes a sufficient number of nucleotides to specifically hybridize to a DNA or RNA target that includes identical or closely related sequences of nucleotides. A probe may contain any number of nucleotides, from as few as about 10 and as many as hundreds of thousands of nucleotides. The conditions and protocols for such hybridization reactions are well known to those of skill in the art, as are the effects of probe size, temperature, degree of mismatch, salt concentration and other parameters on the hybridization reaction. For example, the lower the temperature and higher the salt concentration at which the hybridization reaction is carried out, the greater the degree of mismatch that may be present in the hybrid molecules.

To be used as a hybridization probe, the nucleic acid is generally rendered detectable by labeling it with a detectable moiety or label, such as ³²P, ³H and ¹⁴C, or by other means, including chemical labeling, such as by nick-translation of DNA in the presence of deoxyuridylate biotinylated at the 5′-position of the uracil moiety. The resulting probe includes the biotinylated uridylate in place of thymidylate residues and can be detected [via the biotin moieties] by any of a number of commercially available detection systems based on binding of streptavidin to the biotin. Such commercially available detection systems can be obtained, for example, from Enzo Biochemicals, Inc. [New York, N.Y.]. Any other label known to those of skill in the art, including non-radioactive labels, may be used as long as it renders the probes sufficiently detectable, which is a function of the sensitivity of the assay, the time available [for culturing cells, extracting DNA, and hybridization assays], the quantity of DNA or RNA available as a source of the probe, the particular label and the means used to detect the label.

As used herein, stringency conditions under which DNA molecules form stable hybrids may include:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

or any combination of salt and temperature and other reagents that result in selection of the same degree of mismatch or matching. See, for example Britten et al. Methods Enzymol. 29E: 363-406, 1974.

Further to the identification of the stable integration of the nucleic acid constructs, transcripts of the gene of the pharmaceutical product of interest may be determined by reverse transcription polymerase chain reaction (RT-PCR). With this method, the RNA strand is first reverse transcribed into its DNA complement or complementary DNA, followed by amplification of the resulting DNA using polymerase chain reaction. This can either be a 1 or 2 step process. This approach may be used to detect mRNA transcripts from the second nucleic acid as an indication that the construct present in the transgenic plant cell is expressed.

The pharmaceutical product of interest may be detected by Western blot analysis. This method refers to the analysis of protein (or polypeptides) immobilized onto a support such as nitrocellulose or a membrane. This approach involves first separating a mixture of at least one protein on an acrylamide gel, and then transferring from the gel to a solid support, such as nitrocellulose or a nylon membrane. The immobilized proteins are then exposed to at least one antibody with reactivity against at least one antigen of interest. Bound antibodies are then be detected by various methods, including, but not limited to, the use of radiolabelled or fluorescently-labelled antibodies.

The pharmaceutical product of interest may be detected by ELISA. Originally described by Engvall (Meth Enzymol, 70: 419, 1980), Enzyme-Linked ImmunoSorbent Assay (ELISA) is a biochemical technique used to detect the presence of an antibody or an antigen in a sample.

Another method to identify the protein of interest is by liquid chromatography with tandem mass spectrometry (LC-MS-MS), an analysis approach that combines the solute separation power of HPLC, with the exquisite detection power of a mass spectrometer (van Breemen et al. Expert Opin Drug Metab Toxicol, 1: 175-85, 2005). LC or HPLC can separate peptides on the basis of a number of unique or species specific properties of peptides such as charge, size, hydrophobicity and presence of a specific tag or amino acids. Tandem mass spectrometry (MS-MS) is used to produce structural information about a compound by fragmenting specific sample ions inside the mass spectrometer and identifying the resulting fragment ions. This information can then be pieced together to generate structural information regarding the intact molecule. Tandem mass spectrometry also enables specific compounds to be detected in complex mixtures on account of their specific and characteristic fragmentation patterns. LC-MS-MS may be used to generate primary sequence information from proteins.

A further approach to identify the pharmaceutical product of interest is by gas liquid chromatography (or gas chromatography). This method refers to an approach to separate volatile components of a mixture. A gas chromatograph uses a flow-through narrow tube known as the column, through which different chemical constituents of a sample pass in a gas stream (carrier gas, mobile phase) at different rates depending on their various chemical and physical properties and their interaction with a specific column filling, called the stationary phase. As the chemicals exit the end of the column, they are detected and identified electronically. The function of the stationary phase in the column is to separate different components, causing each one to exit the column at a different time (retention time). Other parameters that can be used to alter the order or time of retention are the carrier gas flow rate, and the temperature. Generally, substances are identified (qualitatively) by the order in which they emerge (elute) from the column and by the retention time of the analyte in the column.

The pharmaceutical product of interest may be assessed functionally, for example, by serum neutralization inhibition assay. Serum neutralization inhibition assay is a serological assay to measure the ability of an antigen to prevent neutralization of a virus by an anti-viral serum. Serum from a host that was immunized with a virus would normally prevent that virus from infecting cells, thus neutralizing the cytopathic effects of the virus. However, if an antigen from that virus was added to the above mixture, then interaction of the antigen with the serum would result in antigen/serum complexes that would prevent the serum from neutralizing the effects of the virus.

Following selection of plant cells based on localization of the gene insert into pericentric rDNA and expression of functional pharmaceutical product of interest, the pharmaceutical product may be recovered from the plants.

The transgenic plant cells are typically harvested, washed and placed in a suitable buffer for disruption. However, the pharmaceutical product may be secreted and thus collected. Further, the pharmaceutical product of interest may be purified once recovered.

The pharmaceutical product of interest may be recovered from cultured plant cells by disrupting cells according to methods known in the art including, but not limited to, mechanical, chemical and enzymatic approaches.

The use of enzymatic methods to remove cell walls is well-established for preparing cells for disruption or for preparation of protoplasts (cells without cell walls) for other uses such as introducing cloned DNA or subcellular organelle isolation. The enzymes are generally commercially available and, in most cases, were originally isolated from biological sources (e.g. lysozyme from hen egg white). Exemplary enzymes include lysozyme, lysostaphin, zymolase, cellulase, mutanolysin, glycanases, proteases, and mannase.

Another method of cell disruption is detergent-based cell lysis. This method may be used in conjunction with homogenization or mechanical grinding. Detergents disrupt the lipid barrier surrounding cells by disrupting lipid:lipid, lipid:protein and protein:protein interactions. The appropriate detergent for cell lysis depends on cell type and source and on the downstream applications following cell lysis. Suitable detergents would be known to the skilled person. Animal, bacterial and plant cells all have differing requirements for optimal lysis due to the presence or absence of a cell wall.

In comparison with ionic detergents, nonionic and zwitterionic detergents are milder, resulting in less protein denaturation upon cell lysis and are often used to disrupt cells when it is critical to maintain protein function or interactions. CHAPS, a zwitterionic detergent, and the Triton™ X series of nonionic detergents are commonly used for cell disruption. Ionic detergents are strong solubilizing agents and tend to denature proteins. SDS is an ionic detergent that is used extensively in studies assessing protein levels by gel electrophoresis and western blotting.

Another method for cell disruption uses small glass, ceramic, or steel beads and a high level of agitation by stirring or shaking of the mix. This method is often referred to as beadbeating. In one aspect, beads are added to the cell or tissue suspension in a test-tube and the sample is mixed on vortex mixer. In another aspect, beadbeating is done in closed vials. The sample and the beads are vigorously in a specially designed shaker driven by an electric motor.

Another method for cell disruption is known as sonication and refers to the application of ultrasound (typically 20-50 kHz) to the sample. In this method, a high-frequency is generated electronically and the mechanical energy is transmitted to the sample via a metal probe that oscillates with high frequency. The probe is placed into the cell-containing sample and the high-frequency oscillation causes a localized low pressure region resulting in cavitation and impaction, ultimately breaking open the cells.

A further method of cell disruption relies on high-shear force. High shear mechanical methods for cell disruption fall into three major classes: rotor-stator disruptors, valve-type processors, and fixed-geometry processors. These processors all work by placing the bulk aqueous media under shear forces that pull the cells apart. These systems are especially useful for larger scale laboratory experiments (over 20 ml) and offer the option for large-scale production. See for example US Patent Application Publication No. US20040268442.

The pharmaceutical product of interest may further comprise a tag, for example, a protein tag. Protein tags find many uses including protein purification, specific enzymatic modification and chemical modification tag. Protein tags are known in the art and include, but are not limited to, affinity tags, solubilization tags, chromatography tags, epitope tags and fluorescent tags. In some instances, these tags are removable by chemical agents or by enzymatic means.

Affinity tags are attached to proteins so that they can be purified using an affinity technique. Exemplary affinity tags include chitin binding protein (CBP), maltose binding protein (MBP), and glutathione-s-transferase (GST). The poly(His) tag binds to metal matrices and is widely-used in protein purification.

Solubilization tags are used to assist in the proper folding in proteins and to prevent protein precipitation. Exemplary solubilization tags include thioredoxin (TRX) and poly(NANP). Some affinity tags may also serve as a solubilization agent, including MBP and GST.

Chromatography tags are used to alter chromatographic properties of the protein to provide different resolution across a particular separation technique. Exemplary chromatography tags include FLAG, consisting of polyanionic amino acids.

Epitope tags are short peptide sequences chosen because of their immunoreactivity with high-affinity antibodies. Exemplary epitope tags include V5-tag, c-myc-tag, and HA-tag. These tags are useful for western blotting, immunoprecipitation and for antibody purification.

Fluorescence tags are used to visualize a protein. GFP and its variants are the most commonly used fluorescence tags.

The recovered pharmaceutical product of interest may be purified. Methods of protein purification are known in the art and include, but are not limited to, centrifugation to separate mixtures of particles of varying masses or densities suspended in a liquid; SDS PAGE to separate proteins according to their size or molecular weight; or by various chromatography approaches including, but not limited to, size exclusion chromatography, ion exchange chromatography, affinity chromatography, metal binding, immunoaffinity chromatography, and high performance liquid chromatography.

The recovered pharmaceutical product of interest finds use depending on the nature of the product. In one aspect, the pharmaceutical product is useful as a vaccinating agent in therapeutic and/or prophylactic immunizations in animals or humans. In another aspect, pharmaceutical product is useful as part of a diagnostic assay.

Uses of the recovered pharmaceutical product of interest are known in the art and include, but are not limited to, vaccination; antibody-based therapeutics for neoplasia, cardiovascular disease, autoimmune disorders, and diabetes; protein hormone replacement or augmentation therapy; and the like.

As used herein, “immunization” and “vaccination” are used interchangeably and refer to a means for providing protection against a pathogen by inoculating a host with an immunogenic preparation containing a pharmaceutical product of interest such that the host immune system is stimulated and prevents or attenuates subsequent pathology associated with the host reactions to subsequent exposures of the pathogen. The pharmaceutical product of interest may be used in conjunction with a pharmaceutically acceptable carrier, diluent or excipient. Pharmaceutically acceptable carriers, diluents and excipients are known in the art and are described, for example, in Remington's Pharmaceutical Sciences 16th edition, Osol, A. Ed. (1980).

A person skilled in the art would know how to prepare suitable vaccine formulations. Conventional procedures and ingredients for the selection and preparation of suitable formulations are described, for example, in Remington's Pharmaceutical Sciences and in The United States Pharmacopeia: The National Formulary (USP 24 NF19) published in 1999.

The forms of the pharmaceutical compositions suitable for injectable use include sterile aqueous solutions or dispersion and sterile powders for the extemporaneous preparation of sterile injectable solutions or dispersions, wherein the term sterile does not extend to any cell that may comprise the pharmaceutical product of interest that is to be administered. In all cases the form must be sterile and must be fluid to the extent that easy syringability exists.

The pharmaceutical product of interest may be useful for the production of therapeutic and/or prophylactic effects in parenteral, oral, mucosal and/or topical applications.

The invention is further illustrated by reference to the following non-limiting examples.

### EXAMPLES

Unless otherwise stated, all DNA manipulations (restriction digests, fragment purification ligation, bacterial transformation and plasmid screening were carried out using standard methods (Sambrook and Russell. Molecular Cloning: A Laboratory Manual (Third Edition) 2001. Cold Spring Harbor Laboratory Press, Cold Spring Harbor, N.Y.).

Purified fragments of the first and second nucleic acids that are free of vector backbone may be used in the context of the invention.

### Example 1

### Nucleic Acids

### First Nucleic Acid

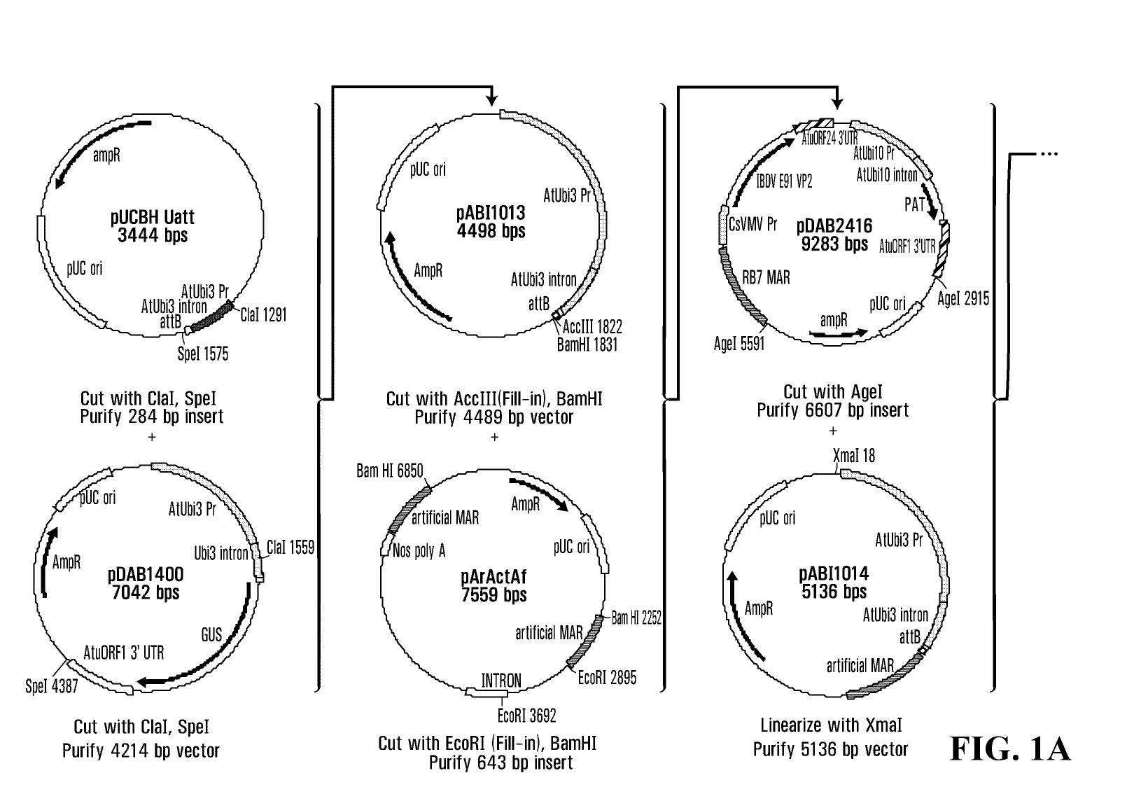

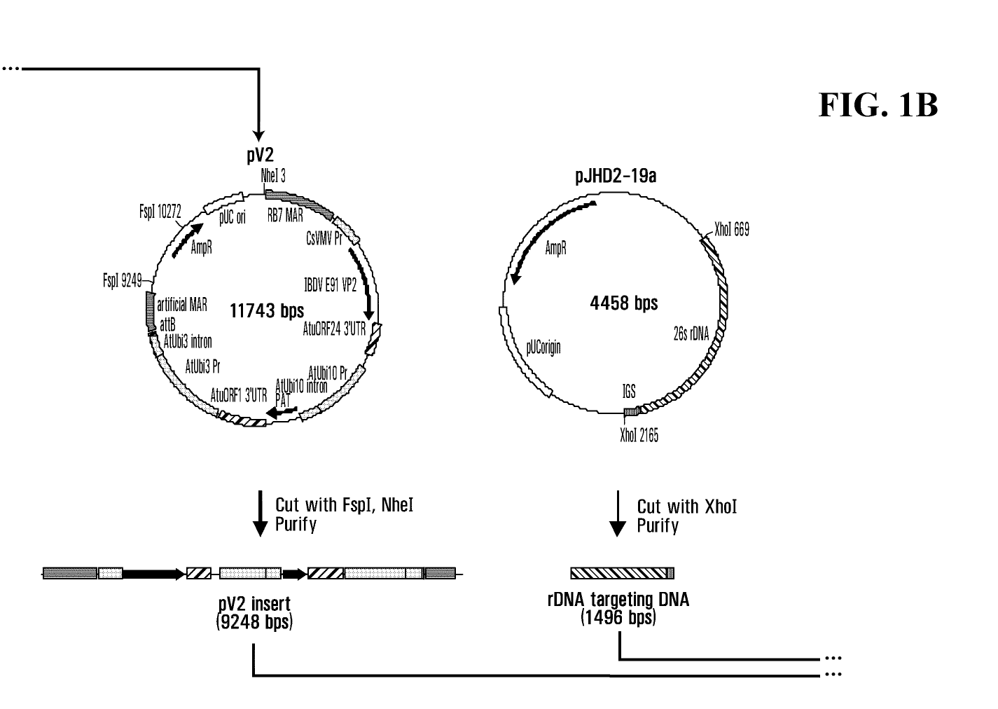

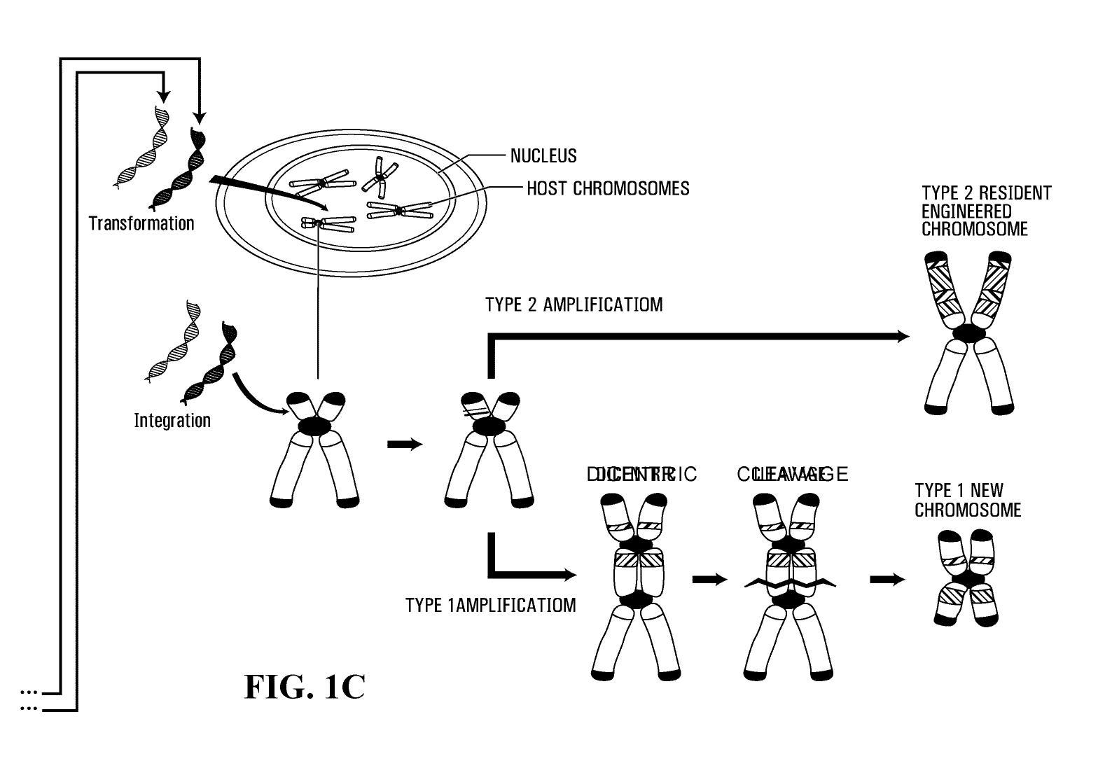

A plasmid containing an Arabidopsis ribosomal RNA gene fragment was constructed by cloning a 1,495 by insert consisting of the 1,384 3′ terminal bases of the 26S Arabidopsis rRNA gene (Unfried and Gruendler. Nucleic Acids Res, 18: 4011, 1990) and the first 111 bases of the 26S-18S rRNA gene intergenic spacer into the XhoI site of pUC9 to generate the vector pJHD19a (FIG. 1B).

### Second Nucleic Acid

For constructing the pV2 vector (maps of intermediate plasmids described below are shown in FIG. 1A), a synthetic fusion sequence comprising the 3′ terminal region of the Arabidopsis thaliana polyubiquitin 3 (AtUbi3) promoter/5′ untranslated region (UTR) (Norris et al. Plant Mol. Biol. 21: 895-906, 1993) and the 33 by E. coli attB A phage insertion site (Landy. Annu Rev Biochem. 58: 913-949, 1989) was synthesized (Blue Heron Technology, Inc) as an insert cloned into a pUC based vector (Bio pUCminusMCS, Blue Heron Technology) to yield the plasmid pUCHUatt. The sequence of the insert is shown below:

(SEQ ID NO: 1)

5′AGATATCGATTCGTAGTGTTTAACATCTGTGTAATTTCTTGCTTGATT
GTGAAATTAGGATTTTCAAGGACGATCTATTCAATTTTTGTGTTTTCTTT
GTTCGATTCTCTCTGTTTTAGGTTTCTTATGTTTAGATCCGTTTCTCTTT
GGAGTTGTTTTGATTTCTCTTACGGCTTTTGATTTGGTATATGTTCGCTG
ATTGGTTTCTACTTGTTCTATTGTTTTATTTCAGGTTGAAGCCTGCTTTT
TTATACTAACTTGAGCGAATCCGGATTAGGATCCGTCGACACTAGTGAAA
GGAGATAGGATCCAAGCTTGGCGTAATCATGGTCATAGCTGTTTCCTGTG
TGAAATTGT 3′

A 284 by ClaI-SpeI fragment consisting of the distal portion of the AtUbi3 promoter/5′ UTR and the attB site was purified from pUCHUatt and cloned into ClaI-SpeI digested plasmid pDAB1400 (containing an expression cassette consisting of the AtUbi3 promoter; the E. coli uidA gene (GUS; Jefferson et al. EMBO J, 6: 3901-3907, 1987) and the Agrobacterium tumefaciens ORF1 3′ UTR (AtuORF1 3′ UTR, Barker et al. Plant Molecular Biology, 2: 335-350, 1983), generating intermediate plasmid pABI013. An artificial matrix attachment region (MAR; van der Geest et al. Plant Biotechnology Journal, 2: 13-26, 2004) was then excised from the plasmid pArActAf as an EcoR1 (Klenow filled)-BamHI fragment and cloned into the AccIII (Klenow filled)-BamHI sites of pABI013, downstream of the attB site, creating intermediate plasmid pABI014. Finally, a 6,607 by Agel fragment from pDAB2406 containing the tobacco RB7 MAR sequence (Hall et al. Proc Natl Acad Sci USA, 88: 9320-9324, 1991) and genes encoding Infectious Bursal Disease Virus (IBDV) E91 VP2 antigen (Tsukamoto et al. Virology, 257: 352-362, 1999) and phosphinothricin acetyl transferase (PAT) (Wohlleben et al. Gene. 70: 25-37, 1988) was cloned into the compatible XmaI site of pABI014 to generate the vector pV2.

Plasmid and fragment purification. Large-scale preparation of pV2 and pJHD19a (rDNA vector) plasmids were carried out from bacterial lysate using Qiagen Giga prep kit (Qiagen) according to the manufacturer's recommended protocol. Prior to transformation, large scale purification of the respective plasmid inserts were carried out (see FIG. 1B) to remove the antibiotic resistance gene using the following procedure. After digestion of the DNA with appropriate restriction enzymes, the DNA was placed at 60° C. for 20 min to inactivate the enzymes. Using HPLC elution buffer (10 mM Tris-HCl pH 8.0, 1 mM EDTA pH 8.0, and 150 mM NaCl), the DNA concentration was adjusted to 1.0-1.2 mg/ml, heated to 55° C. for 10-15 min and 5 mg DNA was loaded at a flow rate of 5 ml/min onto a XK50/100 HPLC column (Amersham) packed with Sephacryl S-1000 SF resin (Amersham). DNA fragments were eluted at a flow rate of 5 ml/min and the elution of DNA fragments were monitored to identify fractions that were free of the fragments containing the vector backbone. The selected fractions were pooled, the DNA concentrated by precipitation and analyzed for purify. The result is a “core pV2” (also referred to as “core pV2 vector”) and a 26S rDNA that are free of their respective vector backbones.

### Example 2

### Tobacco BY-2 Transformation Using Agrobacterium tumefaciens

Tobacco BY-2 cells were transformed with plasmid pDAS1060 by Agrobacterium tumefaciens mediated transfer. The transformation events expressing the highest level of VP2 from 137 calli were advanced to suspension cultures. Transformation events 1060-199, 1060-213 and 1060-243 were selected among the top expressing events from 29 that were analyzed.

### Example 3

### Transformation of Tobacco BY2 Cells with First and Second Nucleic Acids—Protoplast Transformation and Initial Expression Screening

Tobacco BY-2 protoplasts were prepared by a method based on a protocol of Kao and Michyluk (Planta, 126: 105-110, 1975). Approximately three grams of BY-2 suspension culture cells on the third day post subculture were dispensed in a 100×25 mm Petri dish with 25 ml of enzyme solution (1.4% w/v Cellulase ‘Onozuka’ R10, 0.3% w/v Macerozyme R10 in K3 medium with 0.6 M mannitol). K3 medium consists of 2,500 mg/l KNO₃, 250 mg/l NH₄NO₃, 900 mg/l CaCl₂.2H₂O, 250 mg/l MgSO₄.7H₂O, 250 mg/l (NH₄)₂SO₄, 150 mg/l NaH₂PO₄.H₂O, 250 mg/l xylose, 100 mg/l myo-inositol, 1 mg/l pyridoxine-HCl, 10 mg/l thiamine-HCl, 1 mg/l nicotinic acid and 10 ml/l ferrous sulphate/chelate solution (100×; SIGMA F0518), pH 5.8. The dish was sealed with Parafilm and incubated overnight (approximately 16-17 h) at 24-26° C. in the dark with shaking at 50 rpm. The crude protoplast suspension was poured through a 100 μm nylon mesh sieve, 20 ml floating medium (K3 medium supplemented with 0.6 M sucrose) was added and 2.5 ml of W5 solution (154 mM NaCl, 125 mM CaCl₂, 5 mM KCl, 5 mM glucose, pH 5.8) per tube was overlaid. The tubes were centrifuged for 7 min at 115 g and intact protoplasts were harvested from the interface. Protoplasts were washed with 10 ml of W5 solution and then pelleted by centrifugation at 70 g for 7 min. This washing step was repeated twice. Washed protoplasts were resuspended in 5 ml of W5 solution and protoplast concentration determined by counting using a 10 μl haemocytometer (Spore counter, Thoma chamber).

For transformation, protoplasts were resuspended in 3 ml MMM buffer (15 mM MgCl₂, 0.1% w/v 2[N-morpholino]ethanesulfonic acid (MES), 0.5 M mannitol, pH 5.8). Thirty microliters containing 30 μg of DNA was added to 300 μl protoplast solution. 300 μl of PEG solution (40% w/v PEG 4000 in 0.4 M mannitol, 0.1 M Ca (NO₃)₂, pH 6) was then added and the mixture was incubated at room temperature for 20 min. In all experiments, controls were included in which one sample contained neither DNA nor PEG and another sample contained no DNA, but did contain PEG. Subsequently, transfected protoplasts were washed twice by addition of 10 ml W5 per tube followed by centrifugation at 70 g for 7 min. Transformed protoplasts were cultured in XB-4 protoplast medium consisting of 100 ml/l Linsmaier and Skoog Basal Medium 10× stock (PhytoTechnology Laboratories catalog number L689), 0.6 mg/l thiamine-HCl, 170 mg/l KH₂PO₄, 2.5 ml/18P organic acids (100× stock; Kao and Michyluk. Planta, 126: 105-110, 1975), 2.5 ml/18P sugars (100× stock; Kao and Michyluk. Planta, 126: 105-110, 1975), 2 ml/l vitamins (100× stock; Sigma), 5 ml/l coconut water, 0.06 g/l casein hydrolysate, 1 g/l MES, 5 g/l ficoll, 0.2 mg/l 2,4-dichlorophenoxy acetic acid (2,4-D), 0.1 mg/l benzyladenine, 68.4 g/l glucose, 20 g/l sucrose, pH 5.8 at 5×10⁵ protoplasts/ml in 60×15 mm dishes in the dark at room temperature. After 5 to 7 days, the transfected protoplasts were embedded in agarose as follows: melted agarose (2% in medium) (SeaPlaque agarose, Cambrex, catalog number 50100); was cooled to 40-50° C., and mixed with protoplast culture solution to obtain a final agarose concentration of 0.8-1.0% in the solid embedding medium. Ten days post transfection, agarose embedded BY-2 cultures were cut into slices and moved onto new dishes for phosphinothricin (PPT) selection in medium consisting of two parts LS-BY2 (1×LS basal medium diluted from 10× stock (PhytoTechnology Laboratories catalog number L689), 170 mg/l KH₂PO₄, 0.6 mg/l thiamine HCl, 0.2 mg/l 2,4-D, 30 g/l sucrose, pH 5.8) to three parts XB-4 and supplemented with 5 mg/l L-PPT. After 14 days of selection, embedded BY-2 cells were transferred to new dishes in medium consisting of three parts LS-BY2 to two parts XB-4 supplemented with 5 mg/l L-PPT and selection continued for an additional 14 days. When PPT resistant minicalli were observed, the agarose slices were moved onto 100×25 mm dishes containing 0.7% agarose in medium (three parts LS-BY2 to two parts XB-4; 5 mg/l L-PPT) for continued selection. Initial PPT resistant calli were maintained on selection plates until their size reached 3-5 mm at which point they were passaged onto fresh plates as described below.

Callus maintenance and transfer. Callus tissue from the rDNA/core pV2 transformed cells and the VP2/Agrobacterum transformed cells (1060 series transformation events) were subcultured every 14 days throughout the course of the study. Briefly, callus clumps from 14 day old callus were turned over and two to three 5-10 mm diameter sized pieces of callus tissue from the underside of the callus were transferred to a fresh agar plate containing LS-BY2 agar media with bialaphos (10 mg/l) or L-PPT (5 mg/l). The transferred callus pieces were gently pressed onto the agar to ensure good contact with the medium. Each plate was wrapped approximately once with Parafilm, placed in a plastic box and transferred to 25° C.±3° C.

Initiation and maintenance of suspension cultures from callus. Suspension cultures were initiated from callus seven days post transfer. Prior to initiating the suspension cultures, additional callus plates were prepared to obtain sufficient amount of callus. For each transformation event (14-46, 16-37, 16-40, 1060-199, 1060-213 and 1060-243), callus was harvested and mixed as described above. Up to one gram of callus was transferred into a sterile, plastic, 250 ml Erlenmeyer flask containing 50 or 100 ml of LS-BY2 liquid media with bialaphos (10 mg/l) or L-PPT (5 mg/l). The transferred cells and liquid in each flask was drawn up and dispensed five to seven times using a sterile pipette to break up the callus tissue. Three 100 ml suspension cultures were initiated for each event and the sealed flasks were placed in Innova 44 shaker incubators (New Brunswick Scientific) at 25° C.±2° C. in the dark with continuous agitation at 130 rpm with an orbit stroke of 2.54 cm. Unless otherwise noted, all suspension cultures were passaged every seven days by inoculation of fresh media with 0.5% packed cell volume (PCV) of cells.

Callus sample collection and processing. Callus sampling and processing occurred at day seven post callus transfer. For each event, the entire callus was transferred from the medium to the lid of the plate and the callus was stirred into a uniform mixture. Using a one milliliter syringe with the tip cut off, a sample of each callus paste was pulled up to the 0.3 ml division. The cut end of the syringe was placed against the plate lid surface and sample was pushed out to the 0.2 ml division to remove air bubbles. The sample was ejected into a Fastprep Lysing Matrix D sample tube (Q-BIOgene) was extracted immediately or frozen on dry ice prior to storage at −80° C. For extraction, 0.4 ml of phosphate buffer containing 1 mM EDTA (PB/EDTA; 1.5 mM KH₂PO₄, 8 mM Na₂HPO₄, 1 mM EDTA) was added to each tube and placed immediately on ice. The callus samples were processed in a Bio101 Fast Prep cell disruptor (Thermo Savant) for 40 s at a speed of 6.0 followed by a cooling period of approximately three min. This procedure was repeated once followed by centrifugation in an Eppendorf 5415 micro-centrifuge for 15 min at 2,800 g. Sample supernatants were stored at −80° C.±10° C. and the pellets discarded.

Screening of transformed cells in mini-suspension and flask cultures. For initial assessment of tobacco BY-2 clones, 2-3 mm calli were transferred to suspensions in 6 well multi-wells plates containing 3 ml of media plus PPT (referred to as mini-suspension cultures). Water was added outside of the wells to maintain high humidity. Plates were placed in the dark and maintained with shaking at 130 rpm at 26° C. For VP2 analysis, four replicate minicultures were established for each transformation event and minicultures were maintained for 15-20 days prior to harvest and analysis.

At harvest, cell density of mini-suspension cultures was estimated by determining packed cell volume (PCV) as follows: samples were collected using a 10 ml wide-bore pipette, the wells washed sequentially with 3 ml and 5-10 ml medium. Cell suspensions and washings were combined and transferred to a graduated conical tube and the cells were gently pelleted by centrifugation at 200 g for 15 min. Extracts were prepared as per the PM1 protocol (described below) and stored at −20° C. prior to analysis of VP2 and total protein content. Transgenic events expressing high levels of VP2 were transferred to flask cultures and maintained as described below.

### Example 4

### DNA Preparation and Southern Blot Analysis

DNA was isolated from previously frozen (−80° C.) cell pellets. Briefly, when passaging of mini-suspension or flask suspension cultures of L-PPT resistant events (see below), a portion of the cells were pelleted, flash frozen and stored at −80° C. For genomic DNA preparation, frozen pellets (−1 ml packed volume) were transferred to a crucible and ground in liquid nitrogen. The still frozen powder was transferred to a 15 ml conical tube and genomic DNA prepared using the Qiagen DNeasy Plant kit, according to manufacturer's recommended protocol. DNA quantitation was carried out by spectrophotometric determination at OD_{260/280} (Nanodrop model ND-1000).

For Southern blot analysis, 5 to 10 μg of genomic DNA was digested overnight with the appropriate restriction enzyme(s) and then purified by phenol/chloroform extraction followed by ethanol precipitation. Digested samples were fractionated on 0.7% agarose 0.5×TBE (45 mM Tris-borate, 1 mM EDTA, pH 8.0) gels, run overnight at 70 volts (constant voltage). The gel was stained with ethidium bromide and photographed on UV light-box and then treated for 20 min in 0.25 M HCl to depurinate the DNA, followed by a 30 min incubation in 0.4 M NaOH to denature the DNA. DNA was transferred to a TM-XL (Amersham Biosciences) membrane using a Turboblotter apparatus (Schleicher & Schuell Bioscience) and 0.4 M NaOH as the blotting buffer. Blots were hybridized as follows: membranes were pre-incubated in 20 ml of QuikHyb hybridization buffer (Stratagene) plus 100 μg/ml denatured salmon sperm DNA (Invitrogen) overnight at 65° C. in a hybridization oven (Tyler Research Instruments), then pre-hybridization liquid was discarded and replaced with 20 ml of QuikHyb solution supplemented with 100 μg/ml denatured salmon sperm DNA as well as the appropriate heat-denatured probe labeled with ³²P-dCTP (alpha-32 dCTP, Redivue Amersham Bioscience) using random primer labeling (Random Primers DNA labeling System, Invitrogen). Blots were hybridized overnight at 65° C. in a hybridization oven, then washed twice for 15 min per wash in 2×SSC, 0.1% SDS at 65° C. and washed twice for 15 min per wash in 0.1×SSC, 1% SDS at 65° C. Blots were exposed to x-ray film (Hyperfilm ECL, Amersham Biosciences) for periods ranging from 1-3 days.

### Example 5

### Fluorescence In-Situ Hybridization (FISH) Analysis

Suspension cell cultures of the rDNA/core pV2 transformation events were synchronized and blocked at mitosis. Suspension cultures of rDNA/core pV2 transformed cells were sub-cultured as described and allowed to reach stationary phase, at which point they were transferred to an equal volume of fresh MS medium and maintained with shaking for an additional 24 h. To initiate mitotic blocking, Propyzamide (Chem Service, cat no. PS-349) was added to suspension cultures at a final concentration of 30 μM. After 4 h incubation, cells were washed briefly with fresh LS medium. To prepare protoplasts, the blocked cells were immersed in enzyme solution (1 ml packed cell volume to 10 ml enzyme solution containing 1.0% cellulase ‘Onozuka’ RS, 0.0.5% Macerozyme R-10, 0.1% pectinylase Y-23, 130 g/l sucrose, 1.0 g/l CaCl₂.2H₂O, 100 mg/l KH₂PO₄ and 585 mg/l MES, pH 6.0) and incubated at 37° C. for 1.5 h with shaking. The protoplasts were purified by floatation (as described above for protoplast transformation), washed in W5 solution and resuspended by addition of hypotonic swelling medium (25% W5 solution) to a final volume of 14 ml. After incubation in swelling medium for 10 min at room temperature, protoplasts were pelleted by centrifugation at 100 g for 7 min. Finally, the protoplast pellet was fixed by drop-wise addition of ice-cold fixative solution (1 part glacial acetic acid to 3 parts absolute ethanol) with gentle swirling between additions. Fixed cells were stored at −20° C. in fixative solution prior to slide preparation. For preparation of metaphase spreads, a drop of the fixed protoplast suspension in fixative was dispensed from a Pasteur pipette onto a pre-cleaned microscope slide from a distance of approximately 10 cm. After air-drying, the slides were aged at room temperature for at least 24 h prior to hybridization.

FISH probes comprising of purified double-stranded DNA fragments were labeled by nick translation using either biotin-16-dUTP (Enzo Life Sciences) or digoxigenin-11-dUTP (Roche), according to manufacturer's instructions. In some instances, probes were also labeled by PCR reactions in which dTTP was partially replaced by biotin-16-dUTP (1.0 mM, Roche) or digoxigenin-11-dUTP (1.0 mM, Roche). The 26S rDNA probe was a 1.5 kb XhoI fragment of pJHD19a labeled by nick-translation using DIG-Nick Translation Mix (Roche) containing digoxigenin-11-dUTP (Roche), according to manufacturer's instructions. The 18S rDNA probe was prepared by PCR amplification in a reaction containing digoxigenin-11-dUTP (primers used were forward primer: 5′-TGT GCA CCG GTC GTC TCG T-3′ and reverse primer: 5′ TCA GCC TTG CGA CCA TAC T-3′). The pV2 probe consisted of a 9.2 kb NheI-FspI fragment comprising the transfected pV2 insert and was labeled by incorporation of biotin-16-dUTP (Enzo Life Sciences) by nick translation.

Hybridization of the slides with probes was carried out by first incubating the slides in 100 μg/ml RNase A for 1 h at 37° C. and then washing twice for 5 min in 2×SSC. The slides were dehydrated in an ethanol series (70%, 80%, 90% and 100%) for 2 min each at room temperature. The slides were denatured in solution containing 70% formamide and 30% 2×SSC for 2 min at 70° C. then quenched in an ice cold ethanol series (70%, 80%, 90% and 100%) for 2 min each and air dried. After denaturing at 70° C. for 10 min, 200 ng labeled probe mix was added to 26 μl hybridization buffer (50% formamide, 10% (w/v) dextran sulphate, 1% Triton X-100, 2×SSC, pH 7.0) per slide. A coverslip was then placed over the slides, which were then incubated at 37° C. for 16 h in a humidified chamber. Subsequently, the slides were washed in 50% formamide, 2×SSC at 42° C. followed by washing in 2×SSC at 42° C. The slides were then incubated with avidin-FITC (Vector Laboratories) and monoclonal anti-digoxigenin (Sigma) at 37° C. for 30 min followed by a 30 min incubation in biotinylated anti-avidin (Vector Laboratories) and sheep anti-mouse IgG-DIG (Chemicon). The slides were finally incubated in avidin-FITC and anti-dig rhodamine, washed with PBD solution (50 mM phosphate buffer and 0.1% Triton-X 100) and then mounted in DAPI-Vectorshield. The FISH images were captured using an Olympus BX61 microscope equipped for epi-fluorescent illumination (Photonic Solution). The digital images were acquired using FISHView software (Applied Spectral Imaging).

### Example 6

### Characterization of Growth and Stability of Expression and DNA Insert for rDNA/Core pV2 Transformation Events

Suspension cultures were passaged every seven days and subcultured for twelve passages based on % PCV. At passage two, three replicate flasks for each transformation event were expanded to six replicates per event with duplicate flasks being maintained to track passage lineage. Six replicates per event were maintained up to passage eight for the rDNA/core pV2 transformed cells (transformation events 16-xx) and passage 11 for the VP2 Agrobacterium transformed cells (transformation events 1060-xx). At these passages, replicates were reduced to four per event. Due to different growth characteristics determined at the early passages, rDNA/core pV2 transformation events 16-37 and 16-40 were sub-cultured using a 1% final PCV while the remaining events were sub-cultured using a 0.5% final PCV. At day seven post subculture for each passage, the PCV for each flask was determined. For subculturing, the volume of media and amount of packed cells were mixed to obtain a 10% or 5% PCV, followed by a five or ten-fold dilution in the necessary volume of fresh LS-BY2 media resulting in a 1.0% or 0.5% final PCV. Following each subculture, the flasks were placed in Innova 44 shaker incubators at 25° C.±2° C. with continuous agitation at 130 rpm in the dark with an orbit stroke of 2.54 cm.

### Example 7

### Process Method 1 and Total Protein Determination

Process Method 1 (PM1) was used to process all suspension culture samples harvested from samples collected at days 11, 14 and 18 post-inoculation throughout the experiment.

For PM1, each sample, either 10 or 20 ml of suspension were harvested and dispensed into 15 ml conical tubes and centrifuged at 2,000 g for 10 min and 4.0° C. to sediment the cells. The PCV was determined using the graduations on the centrifuge tube to the nearest quarter of one milliliter. The growth media was discarded and each cell pellet was resuspended with a volume of phosphate buffer containing 1 mM EDTA (PB/EDTA; 1.5 mM KH₂PO₄, 8 mM Na₂HPO₄, 1 mM EDTA) equal to the PCV. A volume of approximately 1.3 ml per tube was transferred into Lysing Matrix D sample tubes (Q-BIOgene) and placed immediately on ice. To obtain an adequate amount of cell extract for testing and retention, approximately 6×1.3 ml aliquots were processed per sample. The samples were processed in the Bio 101 Fast Prep cell disruptor (Thermo Savant) for 40 s at a speed of 6.0 followed by a cooling period of approximately three minutes. This procedure was repeated once followed by a centrifugation in an Eppendorf 5415 micro-centrifuge for 15 min at 2,800 g. The supernatants from each tube were decanted into a common pool for each sample and placed immediately on ice. Once all of the processed aliquots for each sample were pooled and vortexed, the samples were dispensed into multiple aliquots and stored at −80° C.±10° C. until analysis.

Prior to analysis at each test point, the conical tubes used for the sample harvest were labeled, pre-weighed and recorded at each passage. After the % PCV was determined, the supernatant was discarded from each sample and the packed cells were place on ice until weighed for determining pellet weight (PW). If pellet weight retainers were required for DNA/RNA analysis, the pelleted samples were frozen at −80° C.±10° C. If wet cake weight (WCW) analysis was required, the samples were placed back on ice for further processing. For samples requiring a wet cake weight determination, sample pellets were resuspended to the original volume of 10 ml by vortexing in deionized water. Each resuspended sample was harvested using 30 μm nylon Spectramesh (Spectrum Laboratories) and subjected to vacuum filtration for approximately 60 s. Each wet cake was rinsed with approximately three volumes of deionized water and the wet cakes were transferred to the pre-weighed aluminum pans and weighed. For each sample, the weight of the sample pan was subtracted from the weight of the pan plus the wet cake to determine the WCW.

Total protein determination. Total soluble protein was determined using the Pierce BCA™ Protein Assay Kit, Microplate Procedure. Serial two-fold dilutions of a bovine serum albumin standard (BSA) diluted to 2 mg/ml and pre-diluted, plant-derived VP2 samples were performed in a 96-well round bottom microtiter plates (BD Falcon). Twenty five microliters of each standard dilution or unknown sample replicate were pipetted into an appropriately labeled 96-well flat bottom microtiter ELISA plate. BCA™ Working Reagent (200 μl/well) was added to each well containing the standard or unknown sample and the plate was mixed on a plate shaker for approximately 30 s. Samples were placed at 37° C. and incubated for 30 min. Plates were cooled to room temperature (approximately 20 min) and the absorbance at OD₅₄₀₋₅₉₀ was measured using a Tecan Sunrise Plate reader. The data were analyzed with Microsoft Office Excel™

### Example 8

### ELISA Analysis for Quantitative Determination of VP2 in Plant Cell Culture Sample Extracts

For quantitation of VP2 by ELISA, Nunc Maxisorp 96-well microtiter ELISA plates were coated with a chicken anti-IBDV polyclonal serum (SPAFAS) diluted in 0.01 M borate buffer and incubated overnight at 2-7° C. The following day, the ELISA plates were removed from 2-7° C. and allowed to equilibrate to room temperature. Plates were washed three times with a wash buffer containing phosphate buffered saline and Tween 20 (PBS-T; 1.5 mM KH2PO4, 8 mM Na2HPO4, 2.7 mM KCl, 137 mM NaCl, 0.05% Tween 20). Following the wash step, plates were blocked with 5% (w/v) non-fat dried milk in PBS-T and incubated for two hours at 37° C. Plates were washed three times with PBS-T. Plant-derived VP2 samples and inactivated IBDV, used as the reference antigen, were pre-diluted in blocking buffer. The reference antigen was diluted to a final concentration of 1 μg/ml VP2. The diluted reference antigen and plant-derived VP2 samples were added to the plate by applying 200 μl of each sample to duplicate wells. Serial two-fold dilutions were made by mixing and transferring 100 μl per well down the plate for a total of six dilutions per reference or sample. Plates were incubated for 1 hr at 37° C. and washed three times with PBS-T. A VP2-specific neutralizing monoclonal antibody (R63, ATCC) was diluted in blocking buffer and added to each plate (100 μl/well) and incubated for 1 h at 37° C. The plates were washed three times with PBS-T. Goat anti-mouse IgG peroxidase-labeled antibody conjugate (KPL) was diluted in blocking buffer and added to each plate (100 μl/well) and the plates were incubated for 1 h at 37° C. Following incubation with conjugate, the plates were washed three times with PBS-T and ABTS (KPL) substrate was added to each plate (100 μl/well). Plates were incubated at room temperature until the 1 mg/ml dilution of reference antigen had reached an OD₄₀₅ absorbance (with a 492 nm reference filter) of 0.8-1.0. The plate was blanked against wells with no antigen and the optical density was determined using a Tecan Sunrise Plate reader. The data was displayed using Tecan Magellan™ Software and exported to Microsoft Excel™ where linear regression and quantitative analysis was performed.

### Example 9

### LC-MS Analysis for Quantitative Determination of VP2 in Plant Cell Culture Sample Extracts

To generate diagnostic peptides suitable for analysis by high performance liquid chromatography with positive-ion electrospray (ESI) tandem mass spectrometry (LC/MSMS), the extracted VP2 protein was digested with the proteinase trypsin. Synthetic peptides were identified according to initial analysis of VP2 protein digests showing which VP2 peptides yield high sensitivity and stability. The corresponding synthetic peptides (SIGMA Genosys) were used to generate a calibration curve for quantitation of VP2 protein from sample extracts. The VP2 tryptic peptide T10 was selected for quantitation and has an amino acid sequence of LGDPIPAIGLDPK and molecular mass of 1305. A T10 stably labeled C13/N15 isotopic peptide (T10IS) was used as an internal standard for all samples tested and has an amino acid sequence of [LC13N15]GD[PC13N15]I[PC13N15]A1G[LC13N15]D[pC13N15]K and molecular mass of 1338.

To prepare the T10IS standard stock solution, 1.5 mg of 13C; 15N T10IS stable isotope labeled synthetic peptide standard was weighed and quantitatively transferred to a one liter volumetric flask and diluted to volume with 50 mM NH₄HCO₃, pH 8.0 to obtain 1.5 μg/ml stable isotope internal standard solution. VP2 peptide calibration standard stock solutions were prepared as follows. One mg of unlabeled T10 VP2 analytical peptide standard was dissolved in 1.0 ml of 50 mM NH₄HCO₃, pH 8.0 to obtain a 1 mg/ml stock calibration standard solution of T10. Ten-fold dilutions of the standard were prepared by serial dilution into the same buffer. VP2 T10 calibration standard solutions over the concentration range 1-6,333 ng/ml were made by diluting the appropriate calibration standard stock solution with 1.0 ml of the 1.5-μg/ml stable isotope solution, then adding 14 ml of 50 mM NH₄HCO₃.

A spin column buffer exchange method was used to transfer VP2 samples into ammonium bicarbonate buffer, which is volatile and can be easily removed for MS analysis. The bottom closure of a Protein Desalting Spin Column (Pierce) was removed and placed into a 2.0 ml microfuge tube. The column was spun at 1,500 g for one minute to remove storage buffer. Eluent storage buffer was discarded and 400 μl of 50 mM NH₄HCO₃ pH 8.0 was added to column and spun at 1,500 g for one minute. This rinse was repeated twice. The spin column was placed into a clean microfuge tube. VP2 extract samples were briefly spun at 20,800 g in a microcentrifuge to remove cellular debris. A 120 μl volume of VP2 sample extracts were then pipetted onto the compacted resin and the column was spun at 1,500 g for 2 min. The sample flow through was collected and this material was used for generation of VP2 tryptic peptides.

For trypsin digestion of VP2 samples, a 100 μl volume of the buffer exchanged VP2 extracts were pipetted into a thin walled microfuge tube. To aid in protein denaturing and trypsin digestion, DTT was added to a 5 mM final concentration and the samples were heated at 95° C. for 20 min and then cooled to 25° C. Trypsin protease (Promega, catalog number V5111) dissolved in 50 mM NH₄HCO₃, pH 8.0 was added to a molar ratio of 20:1 total protein to trypsin enzyme. The samples where incubated at 37° C. for 16 hr and the reaction was terminated with the addition of 15 μl of 10% trifluoroacetic acid. A 9 μl aliquot of the 1.5 μg/ml stable isotope IS solution was added to all samples to serve as internal control. The samples where transferred to an autosamples vial insert and capped for LC-MS analysis.

The series of T10 calibration standards described above were injected using above stated LC-MS conditions. The resulting chromatograms were used to determine peak areas for the ions specific for the VP2 analyte and the T10 internal standard. A standard curve was generated plotting the analyte concentration on the x-axis and the respective IS corrected area on the y-axis. Linear regression analysis was used to determine the equation for the standard curve with respect to the abscissa. The gross concentration of the VP2 T10 peptide in each extract sample was determined by substituting the sample's respective IS corrected T10 peak area into the equation for the calibration curve and calculating peptide concentration.

Mass spectrometry analysis was performed using a MDS/Sciex API 4000 LC/MS/MS system equipped with TurbolonSource source heated to 500° C. MRM scans in negative mode were run at Q1-low, Q3-low resolution with the following parameters: curtain gas (CUR) 45, collision gas (CAD) medium, ion source gas 1 (GS1) 25, ion source gas 2 (GS2) 30. Data was analysed with MDS/Sciex Analyst 1.4.1. The LC system was an Agilent 1100 LC system with a Phenomenex Proteo 2.0×50 mm, 4 μm 90A column heated to 50° C. Injection volumes of 10 μl were used at a flow rate of 500 μl/min. Eluent A was 0.1% (V/V) acetic acid in deionized water and eluent B was 0.1% (V/V) acetic acid in acetonitrile. The gradient used was 0-2 min 5% to 25% B, 2-4 min 25% to 28% B, 5-7 min 100% B, 7.1-10.0 min 5% B.

### Example 10

### Western Blot Analyses of Plant Cell Culture Extracts

For Western blot analysis, samples were diluted to the same total protein concentration then solubilized and denatured with NuPAGE LDS sample buffer, beta-mercaptoethanol and heated for 15 minutes at 94-95° C. Samples containing equal amount of total protein were loaded onto NuPage pre-cast Bis-Tris 12% 10-well mini-gels and the proteins were separated by electrophoresis (Novex X-Cell II Mini-gel apparatus). The fractionated proteins were electrophoretically transferred to 0.2 μM nitrocellulose membranes in a Transblot unit (Novex X-Cell II Blot Module). Membranes were blocked (WesternBreeze Blocker, Invitrogen) and probed with either the VP2-specific monoclonal antibody R63 or a rabbit anti-rVP2 polyclonal antibody raised to purified recombinant VP2 expressed in E coli. Membranes were washed (WesternBreeze Wash Solution, Invitrogen) and probed. Membranes probed with the R63 antibody were incubated with a goat anti-mouse IgG (H+L)−PO₄ conjugate. Membranes probed with the rabbit anti-rVP2 antibody were incubated with a goat anti-rabbit IgG (H+L)−PO₄ conjugate. Following the conjugate incubations, the membranes were washed and incubated with BCIP/NBT substrate (KPL). The reactions were stopped with water. Inactivated IBDV and a plant VP2 reference antigen were included on each gel as positive controls.

### Example 11

### Serum Neutralization Inhibition (SNI) Assay for Plant-Derived VP2 from IBDV

The SNI procedure is a qualitative test to determine the relative inhibition of IBDV binding to anti-IBDV serum by a standardized amount of recombinant VP2 in a serum neutralization assay. Chicken anti-IBDV hyperimmune serum (Charles Rivers Laboratories) was two-fold serially diluted in 96-well cube racks. Cell extracts were diluted to a concentration of 2.0 μg VP/ml based on VP2 quantitative ELISA results. Each pre-diluted sample was mixed in equal volumes with the serially diluted IBDV antiserum and incubated at room temperature for approximately 45 min. The IBDV D78 challenge virus (American Tissue Culture Collection, VR-2041) was diluted to 50-500 TCID₅₀/100 μl and back titered from undiluted to 10⁻³ dilution to ensure the virus was within the correct tissue infective dose range. An equal volume of IBDV 78 challenge was added to each well of the cube racks containing the diluted IBDV antiserum and sample material. The complex was mixed on a plate shaker and incubated at room temperature for approximately 45 min. Growth media was removed from the 96-well plates containing primary chicken embryo fibroblast (CEF) cells (Charles Rivers Laboratories) and samples were inoculated at 4 wells per dilution (200 μl/well). Plates were incubated at 37° C. plus 5% CO₂ for 6±1 days. Plates were observed for cytopathic effect (CPE), and virus infection was confirmed by indirect fluorescent antibody staining. In addition to the rDNA/core pV2 transformation event 14-46 and the VP2 Agrobacterium transformation event 1060-213, a positive control (CVP2-43) was used consisting of tobacco NT-1 cells transformed with the same construct used for the 1060 cell line.

### Example 12

### Transformation of Tobacco BY-2 Plant Cells

The present invention describes the introduction of rDNA/core pV2 to generate transgenic cells comprising core Vp2 integrated into native rDNA array. The locus of gene insertion may be considered an “Engineered Trait Loci” or “ETL” within the context of the present invention. The process by which ETL chromosomes are generated is summarized in FIG. 1C.

Integration of foreign DNA into the rDNA array of host chromosomes may elicit a large scale amplification of the pericentric chromatin (Hadlackzky. Curr Opin Mol Ther 3: 125-132, 2001; Csonka et al. J Cell Sci. 113: 3207-3216, 2000), resulting in dicentric formation and subsequent breakage to yield a fully functional autonomous ETL chromosome (type 1), or a host chromosome with expanded pericentric region and short arm regions (type 2). Apart from the co-amplification of adjacent centromeric sequences in type 1 chromosomes, the large scale amplicon structure is similar between both type 1 and type 2 chromosomes.

A mixture comprising of a second nucleic acid comprising a coding sequence encoding a pharmaceutical product of interest and a 10-fold molar excess of a first nucleic acid consisting essentially of the 26S Arabidopsis rRNA gene (highly homologous to the corresponding N. tabacum rDNA region) was used to transform BY-2 cell protoplasts (FIGS. 1B and 1C) using PEG. The second nucleic acid consists essentially of a “core” pV2 vector (known herein as “core pV2” or “core pV2 vector”) consisting of the coding sequences encoding a herbicide resistant marker gene PAT (Wohlleben et al. Gene. 70: 25-37, 1988), an infectious bursal disease virus (IBDV) VP2 coat protein (Tsukamoto, et al. Virology, 257: 352-362, 1999) and matrix attachment (MARs) sites flanking the VP2 coding sequence to insulate the genes from positional effects (van der Geest et al. Plant Biotechnology Journal, 2: 13-26, 2004; Hall et al. Proc Natl Acad Sci USA, 88: 9320-9324, 1991). VP2 gene expression is driven by the highly expressed cassaya vein mosaic virus (CsVMV) promoter (Verdaguer et al. Plant Molecular Biol, 37: 1055-1067, 1998). VP2 expression levels served as a benchmark for the performance of rDNA/core pV2 transformation events relative to transgenic BY-2 cells transformed with the same VP2 coding sequence using Agrobacterium-based methods.

Transformation events with ETLs containing the VP2 gene were identified using a multi-step screening process. Herbicide resistance transgenic events were screened for VP2 expression. High expressing VP2 events were further characterized by Southern blot analysis to establish the copy number of transgene inserts, and identify those events where a large-scale amplification of the incoming transgene may have occurred. Finally, high copy number, high VP2 expressing cells were characterized at the chromosomal level by fluorescence in-situ hybridization (FISH) to positively identify VP2 integrated chromosomes, i.e., those chromosomes where the transgene was integrated into rDNA arrays of acrocentric chromosome and had elicited a large scale amplification of the pericentric region.

### Example 13

### Expression Screening

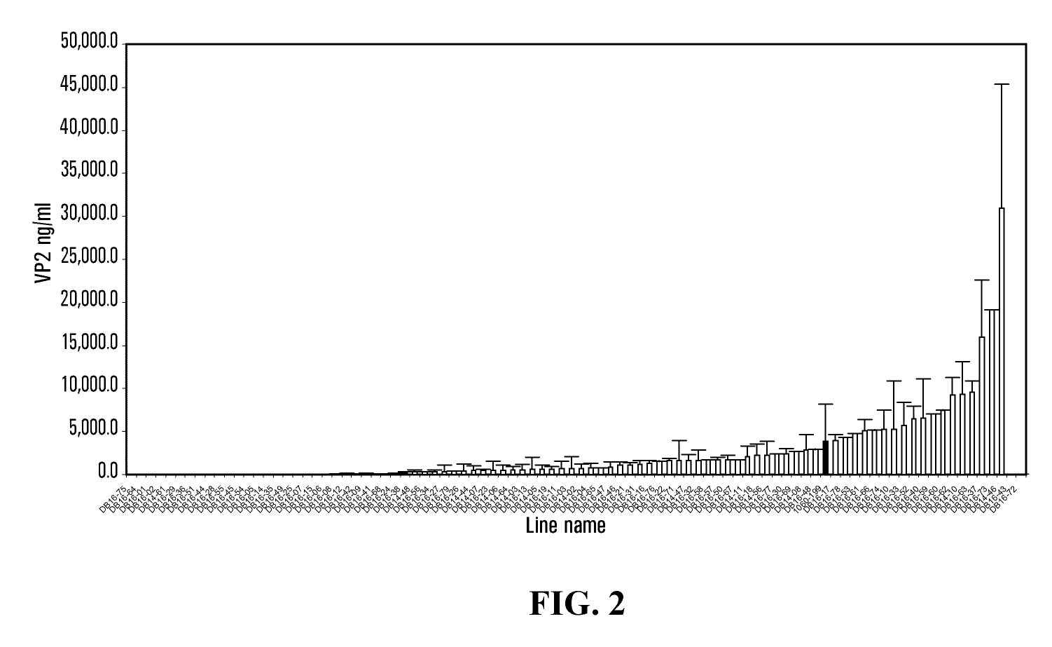

After PPT selection, 105 herbicide resistant transformed calli were obtained and analyzed for VP2 expression. As commercial scale transgenic protein production is normally carried in large batch suspension cultures, the transgenic events were also screened for expression in mini-suspension cultures. For initiating mini-suspension cultures, portions of the calli were manually disaggregated and transferred into media contained in multi-well plates (“mini cultures”). As an internal control as well as for benchmarking purposes, a VP2 expressing BY-2 transgenic cells obtained by Agrobacterium-mediated transformation (1060-199) was cultured and processed in parallel with rDNA/core pV2 transformed cells. As shown in FIG. 2A, 20% of the events expressed VP2 in suspension cultures at levels similar to or greater than the Agrobacterium transformed cells (transformation event 1060-199). Transformation events expressing at or higher levels of VP2 compared to 1060-199 were subsequently transferred into flask scale suspension cultures and maintained according to the protocols described herein for subsequent analysis.

### Example 14

### Molecular Characterization of High Expressing Cells

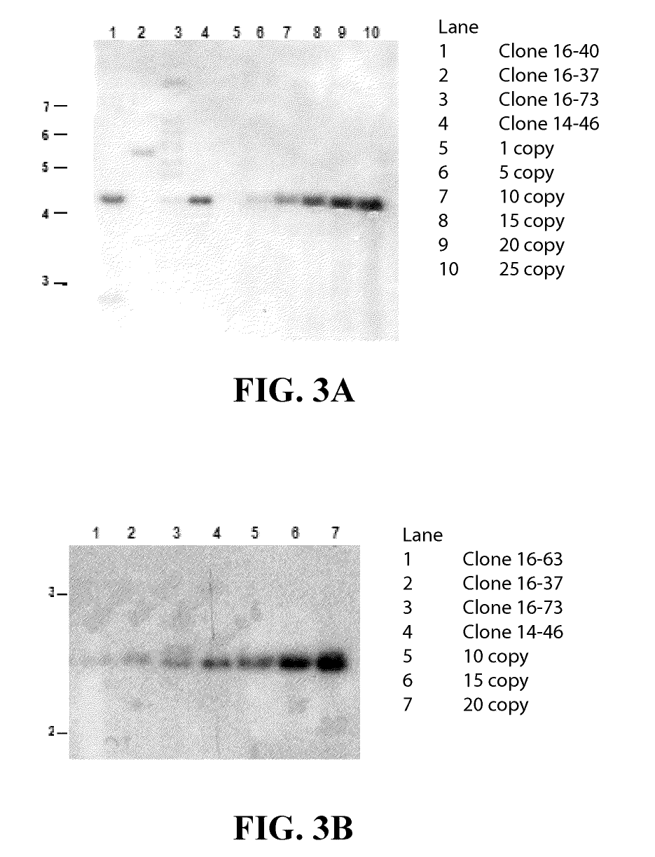

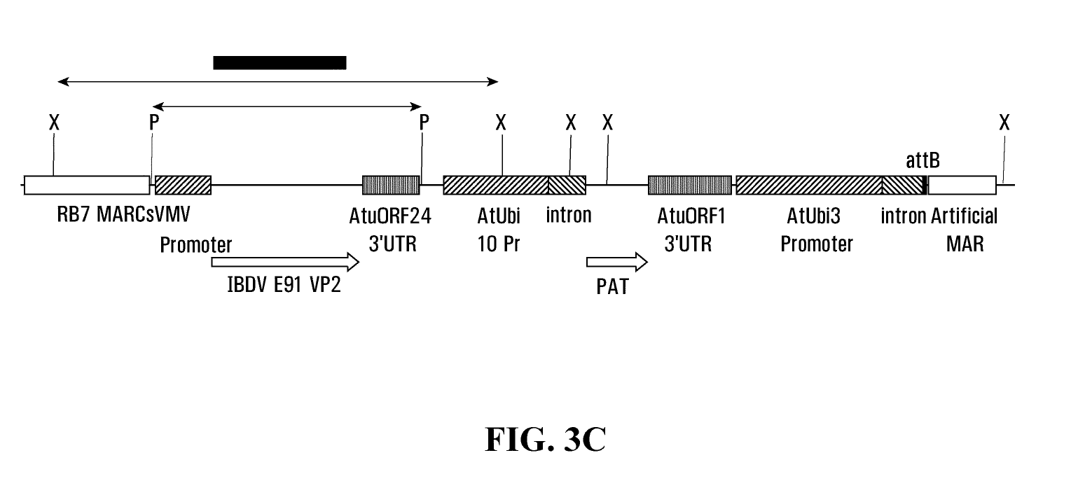

Transgene copy number of transformation events was determined by Southern blots of genomic DNA. Genomic DNA of transformed cell samples were digested by XbaI and hybridized with a ³²P-labeled VP2 fragment probe (FIG. 3A). In several transformation events, bands other than that of the expected size were observed (16-18, 14-56, 14-8, 16-74, 16-10, 16-52). Multiple bands are often indicative of illegitimate recombination into random chromosomal sites and/or rearranged inserts. In other DNA samples, a single band of the predicted molecular weight was observed (14-10, 16-33 16-40 and 14-46), with transgene copy numbers ranging up to 10 copies (14-46). In a few instances, such as that of transformation event 16-37, a single band was observed migrating at a higher apparent molecular weight. Since one of the XbaI sites within the core vector lies close to the 5′ end of the insert, it is possible that one of the XbaI sites was deleted upon integration, resulting in an XbaI fragment larger in size than that predicted. To investigate this possibility, genomic DNA samples were digested with PacI and probed with the VP2 fragment (FIG. 3B). A single band of the predicted size was observed for the 16-37 transformation event, which is consistent with a deletion occurring within the 5′ RB7 MAR region.

### Example 15

### FISH Analysis

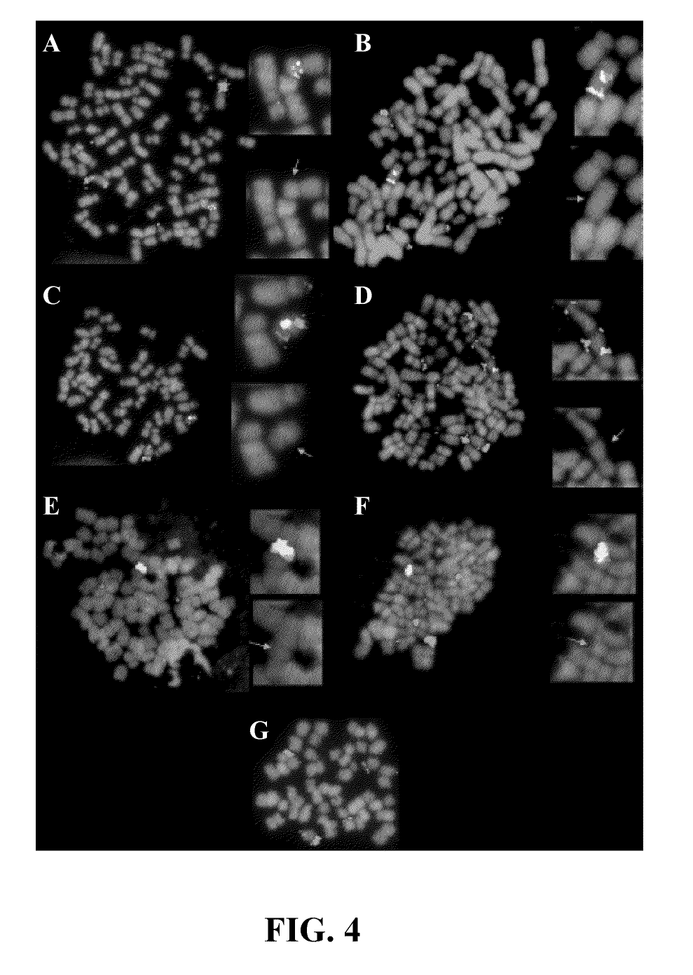

High VP2 expressing (as shown by ELISA in Example 13) and high copy number (as shown by Southern blot in Example 14) transformed cells were subjected to FISH analysis. Metaphase chromosome spreads of transformed cells containing multiple, intact copies of the transgenes were hybridized with a combination of probes directed against core pV2 vector and the native rDNA gene array. The 14-46 transformation event, which expressed VP2 at levels 5-10-fold higher than the VP2 Agrobacterium transformed cells 1060-199 (control), showed a pattern of VP2 hybridization restricted to one small chromosome (see FIG. 4, panels A and B) whereas no staining with core pV2 probes was apparent in the chromosomes of control BY-2 cells (FIG. 4, panel G). The extensive and punctuate labeling of the chromosome by the core pV2 probe is evident in the high magnification images (FIG. 4, panels A and B, insets) and is consistent with a large scale amplification within the region of insertion. The same spreads were co-hybridized with an 18S rDNA probe, whose sequence does not overlap with the 26S rRNA gene used as a carrier in the rDNA/core pV2 transformations. The pattern of 18S rDNA labeling co-localized to the same chromosome containing the integrated core pV2 vector, indicating that core pV2 integrated in an endogenous rDNA locus and that endogenous rDNA repeats were co-amplified along with the core pV2 vector sequences.

The chromosome structure of the 14-46 transformation event may be a type 1 or 2 ETL chromosome. Due to the lack of known pericentric satellite markers in the Nicotiana tabacum, the contribution of pericentric heterochromatin to the chromosome structure of the 14-46 transformation event was not assessed. However, based on observations on mammalian ACE chromosomes (Hadlackzky. Curr Opin Mol Ther, 3: 125-132, 2001) it is likely to be extensive. Given the aneuploid nature of the BY-2 (Lim et al. Chromosoma 109: 161-172, 2000) cell line, no attempts to identify the amplified chromosome in the 14-46 events were made. Of the other high expressing events identified during the initial screen, two others (16-37 and 16-40; FIG. 4, panels C-F) were observed to contain ETL chromosomes, displaying the characteristic targeted integration of core pV2 sequences into endogenous rDNA arrays and subsequent large scale co-amplification of inserted DNA and proximal endogenous rDNA sequences.

Transformation events containing chromosomes in which core pV2 was integrated at rDNA were expanded, were weaned into suspension culture and maintained for multiple passages. As shown in the companion FISH images taken from metaphase chromosomes of cells maintained for 11 additional passages (representing 11 weeks in continuous culture; FIG. 4, panels B, D and F), no apparent change in the chromosome morphology or extent of labeling was observed nor was there any evidence for gross rearrangements or translocations of the amplified regions.

### Example 16

### Comparison of VP2/Agrobacterium Transformed and rDNA/Core pV2 Transformed Tobacco Cell Lines

The rDNA/core pV2 transformation events 14-46, 16-37 and 16-40 were maintained in parallel over multiple passages in suspension culture. Cell line growth, VP2 protein expression levels, stability of VP2 gene expression, stability of core pV2 transgene copy number, VP2 protein quality and biological activity genomic stability were monitored at selected passages. As controls, three VP2 expressing transformed BY-2 cell lines generated by transformation using Agrobacterium tumefaciens (transformation events 1060-199, 1060-213, and 1060-243) were also maintained.

Suspension cultures were initiated from callus tissue. Three flasks of each transformation event were maintained independently. The number of flasks for each rDNA/core pV2 transformed cell line was reduced to two at passage eight. The cell lines underwent 12 passages to determine stability of the core pV2 transgene. VP2 expression of the Agrobacterium transformed cell lines were maintained for five passages. To establish similar growth kinetics in all cultures, the inoculum for transformation events 16-37 and 16-40 was increased two-fold relative to the other events at passage 4. At passage 4, the cultures were considered to be established based on reproducible growth between passages, with the % PCV at time of inoculation (day 7 post-transfer) of that and subsequent passages ranging from 15-40%. Under the growth conditions used, stationary phase was reached at approximately day 11 post-transfer.

For all transformation events, the final % PCVs (measured at day 14 post-transfer) were stable over multiple passages, with percent coefficient of variances between 3 and 13%. The final % PCV values differed slightly among the events, but no pattern was found between the VP2 Agrobacterium transformed and rDNA/core pV2 cell lines when evaluated as groups.

### Example 17

### VP2 Expression

Expression of VP2 in all samples at days 11, 14 or 18 post-transfer was measured by ELISA. As some ELISAs may generate artificially high readings under certain conditions, the amount of VP2 was verified using LC/MS/MS in selected samples. Initial VP2 ELISA data of cells sampled at days 11 and 14 post-transfer established that day 14 post-transfer was consistently higher than day 11 post-transfer and was the peak or close to the peak of expression for the passage (as determined by comparison to day 18 post-transfer samples, data not shown). Day 14 post-transfer samples were therefore used for comparing expression among events and passages.

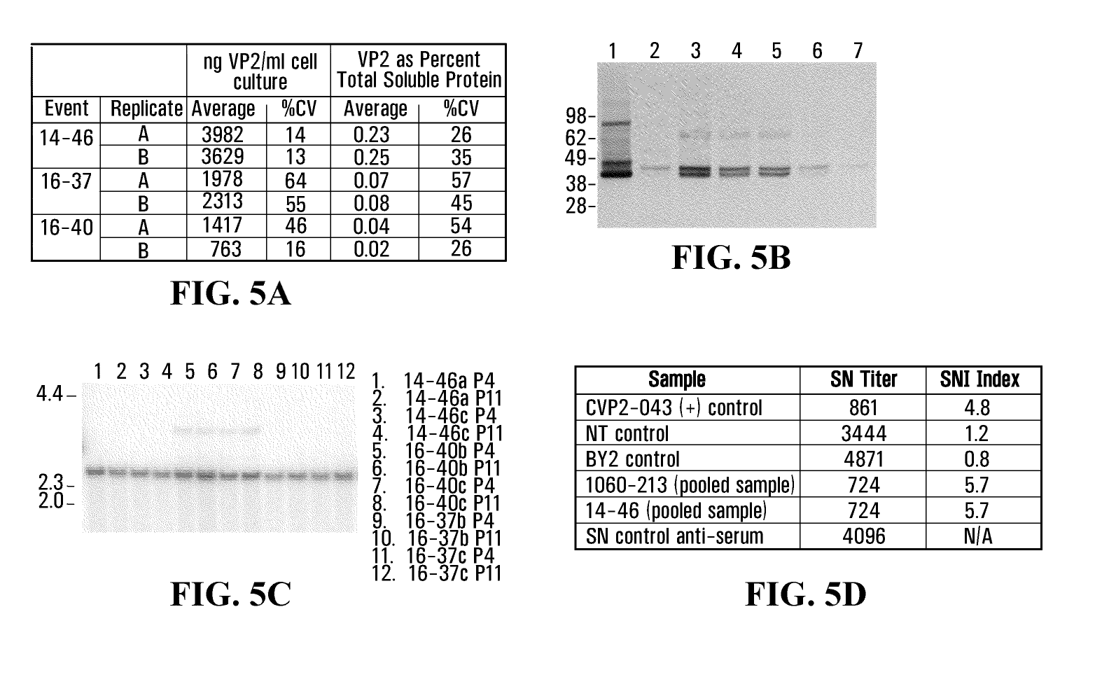

Comparison of VP2 expression levels at early and late passages was carried out for each of the rDNA/core pV2 transformation events. The stability of VP2 expression in independent suspension culture lineages at passages 4-10 were compared (see FIG. 5A). Samples of each transformation event were harvested at Day 14 post-subculturing from three (passages 4-5) or two independent flasks (passages 6-10) and VP2 was quantitated by ELISA. The average and percent coefficient of variance are indicated for ng VP2/ml cell culture and VP2 as a percent of total soluble protein. VP2 expression in all rDNA/core pV2 transformation events were reasonably stable over several passages with event 14-46 showing the highest degree of stability.

The rDNA/core pV2 transformed cells expressed VP2 at a similar level to that of cells transformed by Agrobacterium. The transformation events studied fall into the same statistical grouping when VP2 levels are normalized to either volume of culture or total soluble protein. Similar VP2 levels are found among replicates (three independent lineages for each event). With the exception of the samples for transformation event 16-37, LC/MS/MS data agreed closely with the relative amounts of VP2 contained in the samples, confirming the ELISA results.

Western blot analysis (FIG. 5B) also confirmed the relative expression levels among the events and demonstrated that the protein size and distribution of processed forms of VP2 are the same between the rDNA/core pV2 transformed cells and Agrobacterium transformed cells. A Western blot was probed with a rabbit anti-VP2 antibody to compare VP2 expressed from the nucleic acid constructs of the present invention (lanes 5-7) and by Agrobacterium mediated transformation (lanes 2-4). Processed forms of VP2 seen in the Western blots are similar to those reported in other heterologous expression systems (Lee et al. Biotechnology Progress, 22: 763-769, 2006).

Critical to the use of methods of the invention is the stable integration and expression of the pharmaceutical product of interest. DNA samples from rDNA/core pV2 transformed cells from independent lineages (indicated as a, b or c) of transformation events 14-46, 16-40, and 16-37 were harvested at passage 4 (p4) and passage 11 (p11) were digested with PacI and analyzed by Southern blot. As shown in FIG. 5C, the copy number and integrity of the fragment containing the VP2 coding sequence remains unchanged during this interval. These findings are consistent with the FISH analysis carried out on rDNA/core pV2 transformed cells that was maintained for multiple passages (FIG. 4).

### Example 18

### Serum Neutralization Inhibition Assay

A serum neutralization inhibition (SNI) assay determines the conformational integrity of antigens by evaluating the interference of antigens contained in samples with virus infectivity in a serum neutralization assay. This assay is therefore used to determine the biological equivalence, i.e., the presence of virus neutralizing epitopes of proteins contained in experimental samples. An SNI assay of samples pooled from independent lineages at passage 4 (FIG. 5D) demonstrated that the VP2 produced by the rDNA/core pV2 transformation event 14-46 and the VP2 Agrobacterium mediated transformation event 1060-213 are equivalent in their ability to bind to neutralizing antibodies in sera from IBDV-infected chicken.

### CONCLUSIONS

The studies reported herein demonstrate that ETLs support stable expression of over multiple passages (12) of a biologically active avian viral disease antigen in scalable plant cell cultures. Biological and molecular authenticity of the antigen was confirmed by a variety of means, including ELISA, Western blotting and serum neutralization inhibition assays.

The targeting DNA used was a portion of the 26S rDNA gene coding region to provide a means to favor recombination of the VP2 gene into the rDNA arrays, genomic regions which can naturally amplify. In general, up to 10% of the events recovered contained the VP2 gene localized to the rDNA arrays. The BY-2 Nicotiana cell suspension line was chosen for these experiments for a variety of reasons, including its short doubling time, low level of nicotine and the ability to be scalable to commercial production of proteins of interest.

The examples as described herein compared transformation events produced using Agrobacterium-mediated gene transfer with those generated by the ETL technology (rDNA/core pV2 transformation). Agrobacterium-mediated gene transfer occurs by random insertion of the introduced gene generally into euchromatic genomic regions and is characterized by a large range of expression of the gene of interest among the resultant events. Often events having multiple gene insertions exhibit reduced transgene expression over time, which is generally a result of gene-silencing. Accordingly, obtaining stable and high expressing, multicopy events can be challenging. From the limited number of transformation events described herein it is demonstrated that ETL type events carry multiple copies of the VP2 gene and provide high level and stable gene expression that is similar to Agrobacterium-derived events selected from a much larger pool of events.

The transformation events as described herein showed a pattern typical of ETL chromosome amplifications, with the VP2 gene inserted within large blocks of pericentric heterochromatic rDNA. Molecular analysis suggests that single insertion events are amplified as a result of this process, leading to chromosomal regions that contain multiple copies of the inserted DNA (5-15 copies in this study). FISH analysis confirmed the expected re-iterated periodic structure. Cytological and molecular analysis of these ETL structures before and after the cell lines were placed into culture for multiple passages or under conditions that reflect potential for scale-up to commercial bioreactors showed these structures to be stable.

The initial screening of the transgenic events obtained demonstrated that events with the highest expression typically are those with rDNA-specific genomic amplifications. Within a relatively small number of events (e.g. 100), it is possible to recover a subset that show strong expression when assayed using either callus, mini- or larger-scale suspension cultures. Liquid culture systems were used as a primary screen for transformation events since growth of suspension cultures in bioreactors is the intended commercial platform.

Stability and expression studies included passaging the lead events for at least 12 passages under conditions that are predictive of performance at commercial scale. The results of this analysis indicated that the lead rDNA/core pV2 transformation event 14-46 exhibited a highly stable expression pattern between passages, with a very low level of variation in both expression of the VP2 and performance of the cell lines. The two other rDNA/core pV2 transformation events analyzed (16-37 and 16-40) exhibited a lower level of expression relative to 14-46 and a slightly higher level of variation in cell performance.

Equivalency of key structural components of VP2 protein produced in rDNA/core pV2 transformation event 14-46 and in the high expressing Agrobacterium-derived line (1060-213) was demonstrated. Serum neutralization inhibition assay (SNI) Western blot and ELISA analyses showed that the VP2 protein expressed in the events produced by the different transformation methods are similar in molecular weight and accessibility of a key protective epitope, as demonstrated using a protective epitope-specific monoclonal antibody.

Thus, it is demonstrated that the suspension cultures of ETL transformation events express a biologically active antigen.

The ETL transformation events in BY-2 cell lines described herein meet several criteria for licensure of transgenic plant cell products for animal health products Inherent in any plant-produced product is the lack of animal infectious agents, an important criterion for licensure of animal health products. Other criteria include stability of the gene insertion over multiple passages and a high degree of consistent protein of interest expression and biomass accumulation. Although the number of passages used to assess stability of gene expression and cell growth parameters in this study was relatively small (12), a limited number of passages from the master seed is typical for commercial production of vaccines. Experiments with tobacco cell cultures has shown a high predictability of performance between small scale shake flask suspension cultures and bioreactors, so scalability to volumes necessary for commercial purposes is likely feasible using BY-2 cultures. Together, these characteristics demonstrate that protein production system as described herein could be used to produce commercial animal vaccines.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method for producing a transgenic plant cell culture, comprising:
(a) co-transforming plant cells with:
i. a first nucleic acid, said first nucleic acid comprising a nucleotide sequence of at least contiguous 100 nucleotides, said nucleotide sequence possessing at least 50% sequence identity over its entire length to a native ribosomal DNA (rDNA) sequence of said plant cells; and
ii. a second nucleic acid, said second nucleic acid comprising a coding sequence operably linked to one or more regulatory elements for directing expression of said coding sequence in said plant cells, said coding sequence encoding a pharmaceutical product of interest;
thereby obtaining transgenic plant cells;
(b) culturing a plurality of said transgenic plant cells;
(c) selecting and isolating from said plurality of transgenic plant cells transgenic plant cells wherein said second nucleic acid is stably integrated into or adjacent to native rDNA of said transgenic plant cells and wherein said second nucleic acid is amplified, resulting in said transgenic plant cell culture.

2. The method according to claim 1, wherein step (c) comprises selecting and isolating from said plurality of transgenic plant cells transgenic plant cells wherein said first and second nucleic acids are stably integrated into or adjacent to native rDNA of said transgenic plant cells and wherein said first and second nucleic acids are amplified, resulting in said transgenic plant cell culture.

3. The method according to claim 1, wherein the first and second nucleic acids are on the same construct.

4. The method according to claim 1, wherein the first and second nucleic acids are on different constructs.

5. The method according to claim 1, wherein said second nucleic acid is amplified resulting in 2 to 60 copies of said second nucleic acid.

6. The method according to claim 1, wherein said first nucleic acid is amplified resulting in 2 to 60 copies of said first nucleic acid.

7. The method according to claim 1, wherein a plurality of said second nucleic acids integrate into or adjacent to the native rDNA of said plant cell in sufficiently close proximity to one another that they segregate together as a single genetic locus.

8. The method according to claim 7, wherein one or more of said first nucleic acids integrates into or adjacent to the native rDNA of said plant cell in sufficiently close proximity to said plurality of second nucleic acids that said first and second nucleic acids segregate together as a single genetic locus.

9. The method according to claim 1, wherein said pharmaceutical product of interest comprises an antigen, an antibody, a cytokine, a growth factor, an enzyme, a toxin, a cell receptor, a ligand, a viral or bacterial protein or antigen, a signal transducing agent, or a growth factor.

10. The method according to claim 1, wherein said second nucleic acid comprises a site-specific recombination sequence.

11. The method according to claim 1, wherein said first nucleic acid consists of or consists essentially of said nucleotide sequence possessing at least 50% sequence identity over its entire length to a native ribosomal DNA (rDNA) sequence of said plant cell.

12. The method according to claim 1, wherein the first nucleic acid comprises 5S, 5.8S, 18S or 26S rDNA.

13. The method according to claim 12, wherein the first nucleic acid comprises 26S rDNA.

14. The method according to claim 1, wherein the regulatory element comprises an inducible, constitutive, or tissue specific promoter.

15. The method according to claim 1, wherein the plant cell is a canola cell, a soybean cell, a maize cell, a borage cell, a castor cell, a crambe spp. Cell, a flax cell, a nasturtium cell, an olive cell, a palm cell, a peanut cell, a rapeseed cell, a rice cell, a sunflower cell or a tobacco cell.

16. The method according to claim 15, wherein the plant cell is a tobacco cell.

17. The method according to claim 10, wherein the site-specific recombination sequence is an att sequence, preferably an att sequence of lambda phage.

18. The method according to claim 1, wherein the second nucleic acid sequence comprises a coding sequence that encodes a selectable marker.

19. A transgenic plant cell culture produced by the method according to claim 1.

20. A plant cell obtained from the plant cell culture of claim 19.

21. A transgenic plant cell culture comprising transgenic plant cells comprising a plurality of nucleic acids heterologous to said plant, each of said nucleic acids comprising a coding sequence encoding a pharmaceutical product of interest operably linked to one or more regulatory elements for directing expression of said coding sequence in said plant cell, said nucleic acids being stably integrated at or adjacent to native rDNA of said plant cell.

22. The transgenic plant cell culture according to claim 21, wherein a plurality of said heterologous nucleic acids are integrated at or adjacent to the native rDNA of said plant cell in sufficiently close proximity to one another that they segregate together as a single genetic locus.

23. The transgenic plant cell culture according to claim 21 wherein said heterologous nucleic acid is present in 2 to 60 copies integrated at or adjacent to native rDNA of said plant cell.

24. The transgenic plant cell culture according to claim 21, wherein said plant cells are canola cells, soybean cells, maize cells, borage cells, castor cells, crambe spp. cells, flax cells, nasturtium cells, olive cells, palm cells, peanut cells, rapeseed cells, rice cells, sunflower cells or tobacco cells.

25. The transgenic plant cell culture according to claim 24, wherein said plant cells are tobacco cells.

26. The transgenic plant cell culture according to claim 21, wherein said pharmaceutical product of interest comprises an antigen, an antibody, a cytokine, a growth factor, an enzyme, a toxin, a cell receptor, a ligand, a viral or bacterial protein or antigen, a signal transducing agent, or a growth factor.

27. A plant cell obtained from a plant cell culture according to claim 21.

28. A method for producing a pharmaceutical product of interest, said method comprising:
(a) culturing a transgenic plant cell culture according to claim 19 under conditions sufficient for expression of said pharmaceutical product of interest from said coding sequence; and,
(b) recovering said pharmaceutical product of interest.
